SEQUENCE LISTING (40 sequences)

SEQ ID NO: 1 - protein, 246 aa, Homo sapiens
MAFLRSMWGVLSALGRSGAELCTGCGSRLRSPFSFVYLPRWFSSVLASCPKKPVSSYLRFSKEQLPIFKAQNPDAKTTELIRRIAQRWRELPDSKKKIYQDAYRAEWQVYKEEISRFKEQLTPSQIMSLEKEIMDKHLKRKAMTKKKELTLLGKPKRPRSAYNVYVAERFQEAKGDSPQEKLKTVKENWKNLSDSEKELYIQHAKEDETRYHNEMKSWEEQMIEVGRKDLLRRTIKKQRKYGAEEC

SEQ ID NO: 2 - protein, 42 aa, Homo sapiens
MAFLRSMWGVLSALGRSGAELCTGCGSRLRSPFSFVYLPRWF

SEQ ID NO: 3 - protein, 204 aa, Homo sapiens
SSVLASCPKKPVSSYLRFSKEQLPIFKAQNPDAKTTELIRRIAQRWRELPDSKKKIYQDAYRAEWQVYKEEISRFKEQLTPSQIMSLEKEIMDKHLKRKAMTKKKELTLLGKPKRPRSAYNVYVAERFQEAKGDSPQEKLKTVKENWKNLSDSEKELYIQHAKEDETRYHNEMKSWEEQMIEVGRKDLLRRTIKKQRKYGAEEC

SEQ ID NO: 4 - protein, 346 aa, Homo sapiens
MAASGKLSTCRLPPLPTIREIIKLLRLQAANELSQNFLLDLRLTDKIVRKAGNLTNAYVYEVGPGPGGITRSILNADVAELLVVEKDTRFIPGLQMLSDAAPGKLRIVHGDVLTFKVEKAFSESLKRPWEDDPPNVHIIGNLPFSVSTPLIIKWLENISCRDGPFVYGRTQMTLTFQKEVAERLAANTGSKQRSRLSVMAQYLCNVRHIFTIPGQAFVPKPEVDVGVVHFTPLIQPKIEQPFKLVEKVVQNVFQFRRKYCHRGLRMLFPEAQRLESTGRLLELADIDPTLRPRQLSISHFKSLCDVYRKMCDEDPQLFAYNFREELKRRKSKNEEKEEDDAENYRL

SEQ ID NO: 5 - protein, 396 aa, Homo sapiens
MWIPVVGLPRRLRLSALAGAGRFCILGSEAATRKHLPARNHCGLSDSSPQLWPEPDFRNPPRKASKASLDFKRYVTDRRLAETLAQIYLGKPSRPPHLLLECNPGPGILTQALLEAGAKVVALESDKTFIPHLESLGKNLDGKLRVIHCDFFKLDPRSGGVIKPPAMSSRGLFKNLGIEAVPWTADIPLKVVGMFPSRGEKRALWKLAYDLYSCTSIYKFGRIEVNMFIGEKEFQKLMADPGNPDLYHVLSVIWQLACEIKVLHMEPWSSFDIYTRKGPLENPKRRELLDQLQQKLYLIQMIPRQNLFTKNLTPMNYNIFFHLLKHCFGRRSATVIDHLRSLTPLDARDILMQIGKQEDEKVVNMHPQDFKTLFETIERSKDCAYKWLYDETLEDR

SEQ ID NO: 6 - DNA, 10 bp, Artificial Sequence
  synthetic construct - DNA-binding site of SOX9
agaacaatgg

SEQ ID NO: 7 - protein, 11 aa, artificial sequence
  Synthetic polypeptide - a TAT protein transduction domain
YGRKKRRQRRR

SEQ ID NO: 8 - protein, 9 aa, artificial sequence
  synthetic polypeptide - a protein transduction domain
RKKRRQRRR

SEQ ID NO: 9 - protein, 7 aa, artificial sequence
  Synthetic polypeptide - a protein transduction domain
RRRRRRR

SEQ ID NO: 10 - protein, 12 aa, artificial sequence
  synthetic polypeptide - a protein tranduction domain
RRQRRTSKLMKR

SEQ ID NO: 11 - protein, 27 aa, artificial sequence
  synthetic polypeptide - a protein transduction domain
GWTLNSAGYLLGKINLKALAALAKKIL

SEQ ID NO: 12 - protein, 29 aa, artificial sequence
  synethtic polypeptide - a protein transduction domain
WEAKLAKALAKALAKHLAKALAKALKCEA

SEQ ID NO: 13 - protein, 16 aa, Artificial Sequence
  synthetic polypeptide - a protein transduction domain
RQIKIWFQNRRMKWKK

SEQ ID NO: 14 - protein, 20 aa, artificial sequence
  synthetic polypeptide - an endosomal escape sequence
GDIMGEWGNEIFGAIAGFLG

SEQ ID NO: 15 - protein, 35 aa, artificial sequence
  synthetic polypeptide - a protein transduction domain including an endosomal escape sequence
RRRRRRRRRRRGEGDIMGEWGNEIFGAIAGFLGGE

SEQ ID NO: 16 - protein, 19 aa, artificial sequence
  synthetic polypeptide - predicted sequence from Etfa
MFRAAAPGQLRRAASLLRF

SEQ ID NO: 17 - protein, 35 aa, artificial sequence
  synthetic polypeptide - predicted mitochondrial targeting signal from Dld
MQSWSRVYCSLAKRGHFNRISHGLQGLSAVPLRTY

SEQ ID NO: 18 - protein, 24 aa, artificial sequence
  synthetic polypeptide - a mitochondrial localization signal
MLSRAVCGTSRQLPPVLGYLGSRQ

SEQ ID NO: 19 - protein, 23 aa, artificial sequence
  synthetic polypeptide - a mitochondrial localization signal
LSRAVCGTSRQLPPVLGYLGSRQ

SEQ ID NO: 20 - protein, 24 aa, artificial sequence
  synthetic polypeptide - a mitochondrial localization signal
MLSRAVCGTSRQLAPVLGYLGSRQ

SEQ ID NO: 21 - protein, 23 aa, artificial sequence
  synthetic polypeptide - a mitochondrial localization signal
LSRAVCGTSRQLAPVLGYLGSRQ

SEQ ID NO: 22 - protein, 259 aa, artificial sequence
  synthetic polypeptide - recombinant fusion protein
MARRRRRRRRRRRMAFLRSMWGVLSALGRSGAELCTGCGSRLRSPFSFVYLPRWFSSVLASCPKKPVSSYLRFSKEQLPIFKAQNPDAKTTELIRRIAQRWRELPDSKKKIYQDAYRAEWQVYKEEISRFKEQLTPSQIMSLEKEIMDKHLKRKAMTKKKELTLLGKPKRPRSAYNVYVAERFQEAKGDSPQEKLKTVKENWKNLSDSEKELYIQHAKEDETRYHNEMKSWEEQMIEVGRKDLLRRTIKKQRKYGAEEC

SEQ ID NO: 23 - protein, 258 aa, artificial sequence
  synthetic polypeptide - recombinant fusion protein
ARRRRRRRRRRRMAFLRSMWGVLSALGRSGAELCTGCGSRLRSPFSFVYLPRWFSSVLASCPKKPVSSYLRFSKEQLPIFKAQNPDAKTTELIRRIAQRWRELPDSKKKIYQDAYRAEWQVYKEEISRFKEQLTPSQIMSLEKEIMDKHLKRKAMTKKKELTLLGKPKRPRSAYNVYVAERFQEAKGDSPQEKLKTVKENWKNLSDSEKELYIQHAKEDETRYHNEMKSWEEQMIEVGRKDLLRRTIKKQRKYGAEEC

SEQ ID NO: 24 - DNA, 780 bp, artificial sequence
  synthetic polynucleotide - nucleic acid encoding a recombinant fusion protein
atggcgcgtcgtcgtcgtcgtcgtcgtcgtcgtcgtcgtatggcgtttctccgaagcatgtggggcgtgctgagtgccctgggaaggtctggagcagagctgtgcaccggctgtggaagtcgactgcgctcccccttcagttttgtgtatttaccgaggtggttttcatctgtcttggcaagttgtccaaagaaacctgtaagttcttaccttcgattttctaaagaacaactacccatatttaaagctcagaacccagatgcaaaaactacagaactaattagaagaattgcccagcgttggagggaacttcctgattcaaagaaaaaaatatatcaagatgcttatagggcggagtggcaggtatataaagaagagataagcagatttaaagaacagctaactccaagtcagattatgtctttggaaaaagaaatcatggacaaacatttaaaaaggaaagctatgacaaaaaaaaaagagttaacactgcttggaaaaccaaaaagacctcgttcagcttataacgtttatgtagctgaaagattccaagaagctaagggtgattcaccgcaggaaaagctgaagactgtaaaggaaaactggaaaaatctgtctgactctgaaaaggaattatatattcagcatgctaaagaggacgaaactcgttatcataatgaaatgaagtcttgggaagaacaaatgattgaagttggacgaaaggatcttctacgtcgcacaataaagaaacaacgaaaatatggtgctgaggagtgttaa

SEQ ID NO: 25 - protein, 264 aa, artificial sequence
  synthetic polypeptide - recombinant fusion protein
MRRRRRRRRRRRGEGDIMGEWGNEIFGAIAGFLGGEMLSRAVCGTSRQLPPVLGYLGSRQSSVLASCPKKPVSSYLRFSKEQLPIFKAQNPDAKTTELIRRIAQRWRELPDSKKKIYQDAYRAEWQVYKEEISRFKEQLTPSQIMSLEKEIMDKHLKRKAMTKKKELTLLGKPKRPRSAYNVYVAERFQEAKGDSPQEKLKTVKENWKNLSDSEKELYIQHAKEDETRYHNEMKSWEEQMIEVGRKDLLRRTIKKQRKYGAEEC

SEQ ID NO: 26 - protein, 263 aa, artificial sequence
  synthetic polypeptide - recombinant fusion protein
RRRRRRRRRRRGEGDIMGEWGNEIFGAIAGFLGGEMLSRAVCGTSRQLPPVLGYLGSRQSSVLASCPKKPVSSYLRFSKEQLPIFKAQNPDAKTTELIRRIAQRWRELPDSKKKIYQDAYRAEWQVYKEEISRFKEQLTPSQIMSLEKEIMDKHLKRKAMTKKKELTLLGKPKRPRSAYNVYVAERFQEAKGDSPQEKLKTVKENWKNLSDSEKELYIQHAKEDETRYHNEMKSWEEQMIEVGRKDLLRRTIKKQRKYGAEEC

SEQ ID NO: 27 - DNA, 795 bp, artificial sequence
  synthetic polypeptide - nucleic acid encoding a recombinant fusion protein
atgcggcgacgcagacgtcgtcgtcggcggcgtcgcggcgagggtgatattatgggtgaatgggggaacgaaattttcggagcgatcgctggttttctcggtggagaaatgttatcacgcgcggtatgtggcaccagcaggcagctgcctccagtccttggctatctgggttcccgccagtcatcggtgttagcatcatgtccgaaaaaacctgtctcgtcgtacctgcgcttctccaaagagcagctgccgatttttaaagcgcaaaatccggatgctaaaacgactgaactgattcgccgcattgcacaacgctggcgcgaactcccggacagtaaaaaaaaaatttatcaggacgcctatcgggctgaatggcaggtctataaagaggagatctcacgcttcaaagaacaattaaccccgagtcaaataatgtctctggaaaaagaaatcatggataaacacttaaaacgaaaggcgatgacgaagaaaaaagaactgaccctgctaggtaaacctaagcgtccgcgctctgcgtataatgtgtacgtggcagaacgttttcaggaggccaaaggggattctccgcaagaaaaactgaagaccgtcaaagaaaattggaaaaacctgtctgatagcgaaaaagaactgtacattcagcacgctaaagaagatgagacgcggtatcacaacgaaatgaaatcttgggaagagcagatgatcgaggtcggtcggaaggatcttctccgtcgaaccatcaaaaaacagcgtaaatatggagcagaagagtgctga

SEQ ID NO: 28 - protein, 7 aa, artificial sequence
  synthetic polypeptide - a nuclear localization signal
GKKRSKV

SEQ ID NO: 29 - protein, 7 aa, artificial sequence
  synthetic polypeptide - a nuclear localization signal
KSRKRKL

SEQ ID NO: 30 - protein, 19 aa, artificial sequence
  synthetic polypeptide - a nuclear localization signal
KRPAATKKAGQAKKKKLDK

SEQ ID NO: 31 - protein, 20 aa, artificial sequence
  synthetic polypeptide - a nuclear localization signal
RKKRKTEEESPLKDKAKKSK

SEQ ID NO: 32 - protein, 20 aa, artificial sequence
  synthetic polypeptide - a nuclear localization signal
KDCVMNKHHRNRCQYCRLQR

SEQ ID NO: 33 - protein, 9 aa, artificial sequence
  synthetic polypeptide - a nuclear localization signal
PAAKRVKLD

SEQ ID NO: 34 - protein, 20 aa, artificial sequence
  synthetic polypeptide - a nuclear localization signal
KKYENVVIKRSPRKRGRPRK

SEQ ID NO: 35 - protein, 8 aa, artificial sequence
  synthetic polypeptide - protein purification tag
DYKDDDDK

SEQ ID NO: 36 - protein, 6 aa, artificial sequence
  synthetic polypeptide - protein purification tag
YPYDVP

SEQ ID NO: 37 - protein, 10 aa, artificial sequence
  synthetic polypeptide - protein purification tag
ILKKATAYIL

SEQ ID NO: 38 - protein, 10 aa, artificial sequence
  synthetic polypeptide - protein purification tag
EQKLISEEDL

SEQ ID NO: 39 - protein, 1230 aa, Homo sapiens
MSALCWGRGAAGLKRALRPCGRPGLPGKEGTAGGVCGPRRSSSASPQEQDQDRRKDWGHVELLEVLQARVRQLQAESVSEVVVNRVDVARLPECGSGDGSLQPPRKVQMGAKDATPVPCGRWAKILEKDKRTQQMRMQRLKAKLQMPFQSGEFKALTRRLQVEPRLLSKQMAGCLEDCTRQAPESPWEEQLARLLQEAPGKLSLDVEQAPSGQHSQAQLSGQQQRLLAFFKCCLLTDQLPLAHHLLVVHHGQRQKRKLLTLDMYNAVMLGWARQGAFKELVYVLFMVKDAGLTPDLLSYAAALQCMGRQDQDAGTIERCLEQMSQEGLKLQALFTAVLLSEEDRATVLKAVHKVKPTFSLPPQLPPPVNTSKLLRDVYAKDGRVSYPKLHLPLKTLQCLFEKQLHMELASRVCVVSVEKPTLPSKEVKHARKTLKTLRDQWEKALCRALRETKNRLEREVYEGRFSLYPFLCLLDEREVVRMLLQVLQALPAQGESFTTLARELSARTFSRHVVQRQRVSGQVQALQNHYRKYLCLLASDAEVPEPCLPRQYWEELGAPEALREQPWPLPVQMELGKLLAEMLVQATQMPCSLDKPHRSSRLVPVLYHVYSFRNVQQIGILKPHPAYVQLLEKAAEPTLTFEAVDVPMLCPPLPWTSPHSGAFLLSPTKLMRTVEGATQHQELLETCPPTALHGALDALTQLGNCAWRVNGRVLDLVLQLFQAKGCPQLGVPAPPSEAPQPPEAHLPHSAAPARKAELRRELAHCQKVAREMHSLRAEALYRLSLAQHLRDRVFWLPHNMDFRGRTYPCPPHFNHLGSDVARALLEFAQGRPLGPHGLDWLKIHLVNLTGLKKREPLRKRLAFAEEVMDDILDSADQPLTGRKWWMGAEEPWQTLACCMEVANAVRASDPAAYVSHLPVHQDGSCNGLQHYAALGRDSVGAASVNLEPSDVPQDVYSGVAAQVEVFRRQDAQRGMRVAQVLEGFITRKVVKQTVMTVVYGVTRYGGRLQIEKRLRELSDFPQEFVWEASHYLVRQVFKSLQEMFSGTRAIQHWLTESARLISHMGSVVEWVTPLGVPVIQPYRLDSKVKQIGGGIQSITYTHNGDISRKPNTRKQKNGFPPNFIHSLDSSHMMLTALHCYRKGLTFVSVHDCYWTHAADVSVMNQVCREQFVRLHSEPILQDLSRFLVKRFCSEPQKILEASQLKETLQAVPKPGAFDLEQVKRSTYFFS

SEQ ID NO: 40 - protein, 7 aa, artificial sequence
  synthetic polypeptide - a nuclear localization signal
PKKKRKV

(19) United States
(12) Patent Application Publication
(10) Pub. No.: US 2016/0038565 A1
(43) Pub. Date: Feb. 11, 2016

(54) Methods of Mitigating Side Effects of Radiation Exposure and Chemotherapy

(71) Applicant: Gencia Corporation, Charlottesville, VA (US)

(72) Inventor: Shaharyar Khan, Charlottesville, VA (US)

(21) Appl. No.: 14/408,165

(22) PCT Filed: Jun. 17, 2013
(86) PCT No.: PCT/US13/46133

§ 371 Date: Dec. 15, 2014

Related U.S. Application Data
(60) Provisional application No. 61/660,228, filed on Jun. 15, 2012.
(60) Provisional application No. 61/701,313, filed on Sep. 14, 2012.

(51) Int. Cl. A61K 38/17

(52) U.S. Cl. CPC A61K 38/17

(57) ABSTRACT

Compositions and methods for treating toxicity associated with exposure to radiation and side effects of treatments for hyperproliferative disorders are provided. Typically the compositions are administered in an effective amount reduce one or more adverse side effects. The side effects to be treated include, but are not limited to reduced appetite and weight loss. The methods typically include administering to a subject a composition including a protein transduction domain, a target signal, and a transcription factor A—mitochondrial polypeptide in an amount effective to inhibit, reduce or alleviate weight loss or to increase or induce appetite.

### FIELD OF THE INVENTION

The field of the invention generally relates to methods of reducing side effects of chemotherapy and high levels of radiation by stimulating or enhancing mitochondrial function.

### BACKGROUND OF THE INVENTION

Rapidly dividing cells are sensitive to a number of agents that interfere with cell division. This sensitivity has been exploited by using chemotherapy to treat cancer. Chemotherapy generally refers to the treatment of cancer using one or more toxic antineoplastic agents to kill the rapidly proliferating cancer cells. Most antineoplastic agents are not specific to cancer cells and often kill any rapidly dividing cell including noncancerous, rapidly dividing cells. The death of healthy cells in the patient leads to several unpleasant side effects.

The most common side effects of existing chemotherapies include myelosuppression, alopecia and mucusitis. Additional side effects include depression of the immune system, fatigue, mild to severe anemia, tendency to bleed easily, nausea and vomiting, diarrhea or constipation. Malnutrition and dehydration can cause rapid weight loss, or occasionally weight gain, hair loss and less frequently damage to the heart, liver, kidney, inner ear and brain. Although most of these side effects are transient, some may last for prolonged times or even be permanent. Treatment of the side effects can be accomplished by administering agents such as erythropoietin to increase blood cell counts and antiemetics for treating vomiting and nausea.

Cellular DNA is also susceptible to radiation including ionizing radiation and ultraviolet radiation. Radiation occurs naturally for example as ultraviolet radiation from the sun. Artificial sources of radiation include X-ray machines and particle accelerators. Because radiation disproportionately affects rapidly dividing cells relative to non-proliferative cells, radiation has been used to treat hyperproliferative disorders including cancer.

Radiation therapy can be administered as a therapeutic treatment alone, but is also commonly co-administered with chemotherapy. Although radiation therapy can be targeted to the tissue of interest more easily than chemotherapy, radiation therapy can nonetheless induce severe side effects similar to those resulting from chemotherapy. The most common side effects of radiation therapy are fatigue and skin irritation at the site of treatment; however, other common side effects include mouth and throat sores, intestinal discomfort such as soreness, diarrhea and nausea, swelling and infertility.

Therefore, it is an object of the invention to provide compositions and methods for reducing, inhibiting, or alleviating one or more symptoms associated with side effects of cancer therapies.

It is another object of the invention to provide compositions and methods for reducing, inhibiting, or alleviating one or more side effects associated with, chemotherapy.

It is another object of the invention to provide compositions and methods for reducing, inhibiting, or alleviating one or more side effects associated with, radiotherapy.

It is a further object of the invention to provide compositions and methods for reducing, inhibiting, or alleviating one or more toxic side effects associated with chemotherapy or radiotherapy without reducing the effectiveness of the chemotherapy or radiotherapy to treat cancer.

It is yet another object of the invention to provide compositions and methods for reducing, inhibiting, or alleviating one or more toxic effects associated with exposure to high levels of radiation.

It is yet another object of the invention increase or enhance tolerance of healthy cells to radiation exposure or chemotherapy.

### SUMMARY OF THE INVENTION

Methods and compositions for treating side effects associated with the treatment of hyperproliferative disorders including but not limited to cancer are provided. Methods and compositions for treating radiation exposure are also provided. The methods typically include administering to a subject a composition including a mitochondrial transcription factor or a polynucleotide-binding fragment thereof in an amount effective to inhibit, reduce or alleviate side effects of chemotherapeutic agents, damaging levels of radiation or a combination thereof.

Side effects that can be treated include but are not limited to reduced appetite, weight loss, myelosuppression, mucusitis, low red blood cells count (anemia), fatigue, constipation, diarrhea, nausea and vomiting, bleeding problems, hair loss (alopecia), memory changes, mouth and throat changes, nerve changes, pain, sexual and fertility changes, skin and nail changes, swelling (fluid retention), urination changes (including changes in color and frequency), flu-like symptoms, low infection-fighting white blood cells count (neutropenia), low platelets count (thrombocytopenia), and death.

In some embodiments, the disclosed compositions cause an increase in mitochondrial number, an increase in mitochondrial respiration, increased mitochondrial Electron Transport Chain (ETC) activity, increased oxidative phosphorylation, increased oxygen consumption, increased ATP production, or combinations thereof relative to a control. In some embodiments the composition reduces oxidative stress in the subject.

The compositions can be administered prophylactically or therapeutically. The compositions can be co-administered, or administered in combination with a second therapeutic agent. In some embodiments, the second therapeutic agent includes vitamin supplements, appetite-stimulating medications, medications that help food move through the intestine, nutritional supplements, anti-anxiety medication, anti-depression medication, anti-coagulants, clotting factors, antiemetic medications, antidiarrheal medications, anti-inflammatories, steroids such as corticosteroids or drugs that mimic progesterone, omega-3 fatty acids supplements, steroids, and eicosapentaenoic acid supplements.

Preferably the mitochondrial transcription factor or polynucleotide-binding fragment thereof is part of a fusion protein. The fusion protein can include a protein transduction domain and optionally a targeting signal. A preferred targeting signal is a mitochondrial localization signal, for example, the mitochondrial localization signal of a SOD2 mitochondrial precursor protein. In certain embodiments the polynucleotide-binding polypeptide includes at least one HMG box, such as the HMG box 1 of a transcription factor A mitochondria (TFAM) protein, preferably human TFAM.

### BRIEF DESCRIPTION OF THE DRAWINGS

FIG. 1 is a line graph showing the weight (grams) of mice treated with vehicle, doxorubicin only (DOX), PTD-TFAM only (TFAM), or a combination of PTD-TFAM and doxorubicin (Dox TFAM) as a function of time (days post treatment commencement). Arrows indicate a treatment on day 0, day 4, day 8, and day 12. Error bars indicate Standard Error of the Difference (SED).

FIG. 2A is a line graph showing the daily food consumption (grams) per cage of mice treated with vehicle, doxorubicin only (DOX), PTD-TFAM only (TFAM), or a combination of PTD-TFAM and doxorubicin (Dox TFAM) as a function of time (days post treatment commencement). FIG. 2B is a line graph showing the cumulative food consumption (grams) per cage of mice treated with vehicle, doxorubicin only (DOX), PTD-TFAM only (TFAM), or a combination of PTD-TFAM and doxorubicin (Dox TFAM) as a function of time (days post treatment commencement).

FIG. 3 is a Kaplan-Meier curve showing the fraction of mice surviving as a function of time (days post treatment commencement) after treatment with vehicle, doxorubicin only (Dox), PTD-TFAM only (rhTFAM), or a combination of PTD-TFAM and doxorubicin (Dox-rhTFAM).

FIG. 4 is a Kaplan-Meier curve showing the survival probability (%) of mice as a function of time (days) following treatment with gemcitabine (solid line) and PTD-TFAM in combination with gemcitabine (rhTFAM+Gem) (dashed line) in an in vivo Mia Paca2 SOC combination cancer model.

FIG. 5 is a line graph showing the body weight (average in grams) as function of time (days post treatment commencement) of mice treated with vehicle, gemcitabine, PTD−TFAM only (TFAM) PTD−TFAM in combination with gemcitabine (GEM+TFAM) in an in vivo Mia Paca2 SOC combination cancer model.

FIG. 6 is a Kaplan-Meier curve showing the fraction of C3H/He N mice surviving as a function of time (days post treatment commencement) following lethal dose, 50% (LD50) irradiation and treated with vehicle or rhTFAM.

FIG. 7 is a line graph showing the percent average weight change of C3H/He N mice as a function of time (days post treatment commencement) following lethal dose, 50% (LD50) irradiation and treated with vehicle or rhTFAM.

FIG. 8 is a line graph showing the cumulative food consumption (grams) per cage of C3H/He N mice as a function of time (days post treatment commencement) following lethal dose, 50% (LD50) irradiation and treated with vehicle or rhTFAM.

FIG. 9A-9E are bar graphs showing the % live PanO2 murine pancreatic adenocarcinoma cells following treatment with 10 nM rhTFAM and various doses of Gem: Gemcitabine (FIG. 9A), TMZ: Temozolamide (FIG. 9B), Dox: Doxorubicin (Adriamycin) (FIG. 9C), Cis: Cisplatin (FIG. 9D), or 2-DG: 2-deoxy glucose (FIG. 9E) under hypoxic conditions.

FIG. 10 is a line graph showing mitochondrial membrane potential (JC-1 Aggregate/Monomer (% control)) of “HepG2,” a human hepatocellular carcinoma cell line, with (-x-) or without (-▴-) a 25 μM dose of the chemotherapeutic agent cisplatin, relative to non-malignant fibroblast “Fibro” (-♦-) control cells. The levels are expressed as percentage of control (no rhTFAM added).

FIG. 11 is a line graph showing cell survival (% of control) of non-malignant fibroblast control cells (“Fibro”, -♦-), a human hepatocellular carcinoma cells (“HepG2”, -▴-) and a human hepatocellular carcinoma cells with 25 μM cisplatin (“HepG2+cisplatin”, -X-) after increasing dosages of rhTFAM treatment (μg/ml).

FIG. 12 is a line graph showing the tumor volume (mm³) of HepG2 xenografts over days of treatment with vehicle (-♦-) or 25 μg of rhTFAM (-▪-).

FIG. 13 is a bar graph showing % live of cells untreated (control) or rhTFAM treated cells cultured under hypoxic conditions.

### DETAILED DESCRIPTION

### I. Definitions

In describing and claiming the disclosed subject matter, the following terminology will be used in accordance with the definitions set forth below.

The terms “individual,” “individual,” “subject,” and “patient” are used interchangeably herein, and refer to a mammal, including, but not limited to, humans, rodents, such as mice and rats, and other animals.

As used herein, the term “treating” includes inhibiting, alleviating, preventing or eliminating one or more symptoms or side effects associated with the treatment of hyperproliferative disorders or exposure to damaging levels of radiation.

The term “reduce”, “inhibit”, “alleviate” or “decrease” are used relative to a control. One of skill in the art would readily identify the appropriate control to use for each experiment. For example a decreased response in a subject or cell treated with a compound is compared to a response in subject or cell that is not treated with the compound.

As used herein, “antineoplastic” or “antineoplastic agent” means a substance, procedure, or measure that prevents the proliferation of cells. The term antineoplastic includes, but is not limited to, chemotherapeutics and radiation.

The term “hyperproliferative disorder” refers to a disease, disorder, or syndrome caused by unregulated cell growth or cell division. Exemplary hyperproliferative disorders include but are not limited to diseases related to rapidly proliferating cells such as cancer.

The term “polypeptides” includes proteins and fragments thereof. Polypeptides are disclosed herein as amino acid residue sequences. Those sequences are written left to right in the direction from the amino to the carboxy terminus In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

“Variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of in disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within±2 is preferred, those within±1 are particularly preferred, and those within±0.5 are even more particularly preferred.

Substitution of like amino acids can also be made on the basis of hydrophilicity, particularly, where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamnine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within±2 is preferred, those within±1 are particularly preferred, and those within±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest.

“Identity,” as known in the art, is a relationship between two or more polypeptide sequences, as determined by comparing the sequences. In the art, “identity” also means the degree of sequence relatedness between polypeptide as determined by the match between strings of such sequences. “Identity” can also mean the degree of sequence relatedness of a polypeptide compared to the full-length of a reference polypeptide. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters are used to determine the identity for the polypeptides of the present disclosure.

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

As used herein, the term “low stringency” refers to conditions that permit a polynucleotide or polypeptide to bind to another substance with little or no sequence specificity.

As used herein, the term “purified” and like terms relate to the isolation of a molecule or compound in a form that is substantially free (at least 60% free, preferably 75% free, and most preferably 90% free) from other components normally associated with the molecule or compound in a native environment.

As used herein the term “isolated” is meant to describe a compound of interest (e.g., nucleic acids, polypeptides, etc.) that is in an environment different from that in which the compound naturally occurs, e.g., separated from its natural milieu such as by concentrating a peptide to a concentration at which it is not found in nature. “Isolated” is meant to include compounds that are within samples that are substantially enriched for the compound of interest and/or in which the compound of interest is partially or substantially purified. Isolated nucleic acids or polypeptides are at least 60% free, preferably 75% free, and most preferably 90% free from other associated components.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents.

“Operably linked” refers to a juxtaposition wherein the components are configured so as to perform their usual function. For example, control sequences or promoters operably linked to a coding sequence are capable of effecting the expression of the coding sequence, and an organelle localization sequence operably linked to protein will assist the linked protein to be localized at the specific organelle.

“Localization Signal or Sequence or Domain” or “Targeting Signal or Sequence or Domain” are used interchangeably and refer to a signal that directs a molecule to a specific cell, tissue, organelle, intracellular region or cell state. The signal can be polynucleotide, polypeptide, or carbohydrate moiety or can be an organic or inorganic compound sufficient to direct an attached molecule to a desired location. Exemplary targeting signals include mitochondrial localization signals from the precursor proteins list in U.S. Pat. No. 8,039,587, and cell targeting signals known in the art such as those in Wagner et al., Adv Gen, 53:333-354 (2005) the disclosures of which are specifically incorporated by reference herein in their entireties. It will be appreciated that the entire sequence need not be included, and modifications including truncations of these sequences are within the scope of the disclosure provided the sequences operate to direct a linked molecule to a specific cell type. Targeting signals of the present disclosure can have 80 to 100% sequence identity to the mitochondrial localize signal or cell targeting signal sequences. One class of suitable targeting signals include those that do not interact with the targeted cell in a receptor:ligand mechanism. For example, targeting signals include signals having or conferring a net charge, for example a positive charge. Positively charged signals can be used to target negatively charged cell types such as neurons and muscle. Negatively charged signals can be used to target positively charged cells.

“Tropism” refers to the propensity of a molecule to be attracted to a specific cell, cell type or cell state. In the art, tropism can refer to the way in which different viruses and pathogens have evolved to preferentially target to specific host species, or specific cell types within those species. The propensity for a molecule to be attracted to a specific cell, cell type or cell state can be accomplished by means of a targeting signal.

“Cell Type” is a manner of grouping or classifying cells in the art. The term cell type refers to the grouping of cells based on their biological character determined in part through common biological function, location, morphology, structure, expression of polypeptides, nucleotides or metabolites.

“Cell State” refers to the condition of a cell type. Cells are dynamic throughout their life and can achieve various states of differentiation, function, morphology and structure. As used herein, cell state refers to a specific cell type throughout its lifetime.

“Cell surface marker” refers to any molecule such as moiety, peptide, protein, carbohydrate, nucleic acid, antibody, antigen, and/or metabolite presented on the surface or in the vicinity of a cell sufficient to identify the cell as unique in either type or state.

### II. Methods

Methods for treating one or more symptoms or side effects associated with exposure to radiation or treatment for a hyperproliferative disorder are described. Suitable compositions for use with the disclosed methods are discussed in detail below and typically include an effective amount of a mitochondrial DNA-binding polypeptide, preferably a mitochondrial transcription factor or polynucleotide-binding fragment thereof. Examples mitochondrial DNA-binding polypeptides include, but are not limited to, mitochondrial transcription factors such as transcription factor A, mitochondrial (TFAM), transcription factor B1, mitochondrial (TFB1M), transcription factor B2, mitochondrial (TFB2M), Polymerase (RNA)

Mitochondrial (DNA directed) (POLRMT); and functional fragments, variants, and fusion polypeptides thereof.

Exemplary fusion proteins containing a mitochondrial transcription factor polypeptide are disclosed in U.S. Pat. Nos. 8,039,587, 8,062,891, 8,133,733, and U.S. Published Application Nos. 2009/0123468, 2009/0208478, and 2006/0211647 all of which are specifically incorporated by reference herein in their entireties.

In preferred embodiments the composition includes a mitochondrial DNA-binding polypeptide. The mitochondrial DNA-binding polypeptide can be a recombinant fusion protein including a mitochondrial DNA-binding polypeptide, a protein transduction domain, and optionally one or more targeting signals. In some embodiments, the disclosed compositions cause an increase in mitochondrial number, an increase in mitochondrial respiration, an increase mitochondrial Electron Transport Chain (ETC) activity, increased oxidative phosphorylation, increased oxygen consumption, increased ATP production, or combinations thereof relative to a control. In preferred embodiments, the disclosed methods cause a reduction in oxidative stress in the subject compared to a control.

A. Methods of Treatment

The disclosed compositions can be used to reduce, inhibit, or alleviate one or more toxic or undesirable side effects associated with the administration of an antineoplastic agent. Antineoplastic agents include chemotherapeutic agents and radiation.

The composition can be administered prophylactically, therapeutically, or combinations thereof. Therefore, the composition can be administered during a period before, during, or after administration of or exposure to the antineoplastic agent, or any combination of periods before, during or after administration of or exposure to the antineoplastic agent. For example, the subject can be administered the composition 1, 2, 3, 4, 5, 6, or more hours, or 1, 2, 3, 4, 5, 6, 7, or more days before administration of or exposure to an antineoplastic agent. In some embodiments, the subject can be administered one or more doses of the composition every 1, 2, 3, 4, 5, 6 7, 14, 21, 28, 35, or 48 days prior to a first administration of or exposure to the antineoplastic agent.

The subject can also be administered the composition for 1, 2, 3, 4, 5, 6, or more hours, or 1, 2, 3, 4, 5, 6, 7, or more days after administration of or exposure to an antineoplastic agent. The subject can also be administered the composition during administration of or exposure to an antineoplastic agent. The composition can be administered on the same day as the antineoplastic agent, or on a different day. The subject can be administered one or more doses of the composition every 1, 2, 3, 4, 5, 6, 7, 14, 21, 28, 35, or 48 days during or after administration of the antineoplastic agent.

The compositions can be administered as part of therapeutic regime for the treatment of cancer. For example, if the antineoplastic agent is administered to a subject every fourth day, the composition can be administered on the first, second, third, or fourth day, or combinations thereof. The composition may be repeatedly administered throughout the entire antineoplastic agent administration regiment. In preferred embodiments, the compositions and methods disclosed herein reduce the toxicity or side effects of the antineoplastic agent without reducing or eliminating its ability to effectively treat cancer.
1. Methods for Treating Side Effects Associated with Therapeutic Administration of an Antineoplastic Agent

“Therapeutic administration” or “administration of a therapeutically effective dose” of an antineoplastic agent is a dose that is effective to treat one or more symptoms of the disease to be treated. For example, if the antineoplastic agent is administered to a subject with cancer, a therapeutic dose is a dose effective to treat one or more symptoms of the cancer, for example, reduce tumor burden for reduce tumorigenesis.

Typically the disclosed compositions are administered to a subject in need thereof in an amount effective to inhibit or reduce one or more side effects associated with administration of an antineoplastic agent compared to a control. Common side effects accompanying administration of a therapeutic dose of an antineoplastic agent include reduced appetite (i.e., food intake), weight loss, myelosuppression, mucusitis, low red blood cells count (anemia), fatigue, constipation, diarrhea, nausea and vomiting, bleeding problems, hair loss (alopecia), infection, memory changes, mouth and throat changes, nerve changes, pain, sexual and fertility changes, skin and nail changes, swelling (fluid retention), urination changes (including changes in color and frequency), flu-like symptoms, low infection-fighting white blood cells count (neutropenia), low platelets count (thrombocytopenia). Other side effects include malnutrition, dehydration, damage to the heart, liver, kidney, inner ear and brain, abdominal pain, impotence, acid indigestion, incoordination, acid reflux, infection, allergic reactions, injection site reactions, alopecia, injury, anaphylasix, insomnia, anemia, iron deficiency anemia, anxiety, itching, lack of appetite, arthralgias, asthenia, joint pain, ataxia, azotemia, kidney problems, balance & mobility changes, bilirubin blood level, leukopenia, bone pain, loss of libido, bladder problems, liver dysfunction, bleeding problems, liver problems, blood clots, loss of libido, blood pressure changes, low blood counts, blood test abnormalities, low blood pressure (hypotension), breathing problems, low platelet count, bronchitis, low red blood cell count, bruising, low white blood cell count, lung problems, cardiotoxicity, cardiovascular events, memory loss, cataracts, menopause, central neurotoxicity, metallic taste, chemo brain, mouth sores, chest pain, mucositis, chills, muscle pain, cognitive problems, myalgias, cold symptoms, myelosuppression, confusion, myocarditis, conjunctivitis (pink eye), constipation, cough, nail changes, cramping, nausea, cystitis, nephrotoxicity, nervousness, neutropenia, deep vein thrombosis (DVT), neutropenic fever, dehydration, nosebleeds, depression, numbness, diarrhea, dizziness, drug reactions, ototoxicity, dry eye syndrome, dry mouth, dry skin, pain, dyspepsia, palmar-plantar erythrodysesthesia (PPE), dyspnea, pancytopenia, pericarditis, peripheral neuropathy, early satiety, pharyngitis, edema, photophobia, electrocardiogram (ECG/EKG) changes, photosensitivity, electrolyte imbalance, pneumonia, esophagitis, pneumonitis, eye problems, post-nasal drip, proteinuria, pulmonary embolus (PE), fatigue, pulmonary fibrosis, feeling faint, pulmonary toxicity, infertility, fever, flatulence, radiation recall, flu-like syndrome, rash, flushing, rapid heart beat, rectal bleeding, restlessness, gas, rhinitis, gastric reflux, ringing ears, gastroesophageal reflux disease (GERD), runny nose, genital pain, granulocytopenia, gynecomastia, sadness, glaucoma, seizures, safety, hyposexuality, shortness of breath, hair loss, sinusitis, hand-foot syndrome, skin reactions, headache, sleep problems, hearing loss, sore mouth, hearing problems, stomach sour, heart failure, stomach upset, heart palpitations, stomatitis, heart problems, swelling, heart rhythm changes, heartburn, hematoma, taste changes, hemorrhagic cystitis, thrombocytopenia, hepatotoxicity, low thyroid hormone levels, high blood pressure (hypertension), tingling, high liver enzymes, tinnitus, hyperamylasemia (high amylase), trouble sleeping, hypercalcemia (high calcium), hyperchloremia (high chloride), hyperglycemia (high blood sugar), urinary tract infection, hyperkalemia (high potassium), hyperlipasemia (high lipase), hypermagnesemia (high magnesium), vaginal bleeding, hypernatremia (high sodium), vaginal dryness, hyperphosphatemia (high phosphous), vaginal infection, hyperpigmentation, vertigo, hypersensitivity skin reactions, vomiting, hypertriglyceridemia (high triglycerides), hyperuricemia (high uric acid), hypoalbuminemia (low albumin), water retention, hypocalcemia (low calcium), watery eyes, hypochloremia (low chloride), weakness, hypoglycemia (low blood sugar), weight changes, hypokalemia (low potassium), weight gain, hypomagnesemia (low magnesium), weight loss, hyponatremia (low sodium), hypophosphatemia (low phosphous) and xerostomia. In the most preferred embodiments, the composition is administered in an effective amount to increase appetite (i.e., food intake), reduce weight loss, death or combinations thereof.
a. Chemotherapeutic agents

In some embodiments, the antineoplastic agent is a chemotherapeutic drug. The majority of chemotherapeutic drugs can be divided in to: alkylating agents, antimetabolites, anthracyclines, plant alkaloids, topoisomerase inhibitors, antineoplastic antibiotics, and other antitumour agents. All of these drugs affect cell division or DNA synthesis and function in some way. Representative chemotherapeutic agents include, but are not limited to alkylating agents (such as cisplatin, carboplatin, oxaliplatin, mechlorethamine, cyclophosphamide, chlorambucil, dacarbazine, lomustine, carmustine, procarbazine, chlorambucil and ifosfamide), antimetabolites (such as fluorouracil (5-FU), gemcitabine, methotrexate, cytosine arabinoside, fludarabine, and floxuridine), antimitotics (including taxanes such as paclitaxel and decetaxel and vinca alkaloids such as vincristine, vinblastine, vinorelbine, and vindesine), anthracyclines (including doxorubicin, daunorubicin, valrubicin, idarubicin, and epirubicin, as well as actinomycins such as actinomycin D), cytotoxic antibiotics (including mitomycin, plicamycin, and bleomycin), and topoisomerase inhibitors (including camptothecins such as irinotecan and topotecan and derivatives of epipodophyllotoxins such as amsacrine, etoposide, etoposide phosphate, and teniposide).

In some embodiments, the compositions disclosed herein are administered in an effective amount to prevent, reduce, alleviate, or inhibit one or more side effects of chemotherapy, such as those discussed. The most common side effects of chemotherapy include myelosuppression, alopecia and mucusitis. Other common side effects include depression of the immune system, fatigue, mild to severe anemia, tendency to bleed easily, nausea and vomiting, diarrhea or constipation.
b. Radiation Therapy

In some embodiments the antineoplastic agent is radiation therapy (also referred to as radiation oncology, or radiotherapy, and sometimes abbreviated to XRT or DXT). Radiation therapy is the medical use of radiation, typically ionizing radiation and includes, but is not limited to, external beam radiation, brachytherapy, and radioisotope therapy. Radiation therapy can be used as part of cancer treatment regime to control or kill malignant cells. Radiation therapy also has several applications in non-malignant conditions, including but not limited to treatment of trigeminal neuralgia, thyroid eye disease, pterygium, pigmented villonodular synovitis, and prevention of keloid scar growth, vascular restenosis, and heterotopic ossification.

In some embodiments, radiation therapy includes directing shaped radiation beams at a target tissue, such as the site of a tumor, to maximize the radiation dose to the target tissue while minimizing exposure to the surrounding health tissue. Alternatively, total body irradiation (TBI) is a radiation therapy technique used to prepare the body to receive a bone marrow transplant.

Radiation therapy can be administered with surgery, chemotherapy, hormone therapy, immunotherapy or combination thereof. Radiation therapy is particularly effective when administered in combination with a chemotherapeutic drug. Therefore, in some embodiments the antineoplastic agent is a combination of chemotherapy and radiation therapy. Radiation therapy can be administered before, during, or after chemotherapy, or combinations therof.

The amount of radiation used in photon radiation therapy is typically measured in gray (Gy), and can vary depending on the target tissue, the disease to be treated, the desired outcome, and the source of radiation. Common radiation dosages are known or can be determined by one of skill in the art based on the patient and condition to be treated. Dosages can range from less than 1 Gy to more than 2000 Gy, but are typically between about 20 Gy and 100 Gy. For example, a dose administered to ablate a solid epithelial tumor can range from 60 to 80 Gy, while lymphomas can be 20 to 40 Gy, and preventative (adjuvant) doses can be about 45-60 Gy in 1.8-2 Gy fractions depending on the cancer to be treated.

In some embodiments, the compositions disclosed herein are administered in an effective amount to prevent, reduce, alleviate, or inhibit one or more side effects of radiation therapy or radiation exposure. The most common side effects of radiation therapy or radiation exposure are fatigue and skin irritation at the site of radiation. Other common side effects are very similar to those observed in chemotherapy and include mouth and throat sores, intestinal discomfort such as soreness, diarrhea and nausea, swelling and infertility.
2. Methods of Treating Occupational, Accidental, or Weapon-Induced Exposure to Radiation

In some embodiments, the compositions disclosed herein are used to prevent, reduce, alleviate, or inhibit one or more symptoms associated with occupational, accidental, or weapon-induced exposure to damaging levels of radiation. The exposure can be acute or chronic. The exposure can be direct from the source, or indirect, such as from contaminated radioactive material.

The individual is typically exposed to a level of radiation above background levels. The level of radiation is typically high, for example greater than 50 mSv per year. The individuals can be treated prophylactically, prior to exposure to the antineoplastic agent, or therapeutically after exposure, or combinations thereof.

Exposure to radiation can be a result of occupational exposure, an accident, or an act of war or terrorism. Occupational exposure to radiation is typical of industries including, but not limited to, airline, industrial radiography, medical radiology and nuclear medicine, mining of uranium and other radioactive isotopes, nuclear power plant and nuclear fuel reprocessing, and laboratory research. Examples of subjects experiencing accidental exposure include first responders to radiation incidents such as nuclear power accidents or acts of terror, or in the event of war. The general population could also be exposed to levels of radiation as a result of an accident, such destruction or meltdown of a nuclear reactor, or an act of war, such as fallout from the use of nuclear weapons. It is believed that individuals downwind of a nuclear event such as nuclear weapon detonation could be exposed to doses of radiation that would exceed 300 Gy per hour. As a reference, 4.5 Gy (around 15,000 times the average annual background rate) is fatal to half of a normal population, without medical treatment.

Exposure to radiation can lead to acute radiation syndrome (abbreviated ARS, and also referred to as radiation sickness), poisoning, and death. ARS can occur after most or all of an individual's body is exposed to radiation. The exposure is typically a high level of radiation received in short period of time, for example, within minutes. Doses from medical procedures such as chest X-rays are typically too low to cause ARS, however, doses from radiation therapy to treat cancer may be high enough to cause some ARS symptoms.

In some embodiments, the compositions disclosed herein are administered in an effective amount to prevent, reduce, alleviate, or inhibit one or more side effects of ARS. Symptoms can be similar to those of radiotherapy and chemotherapy and include hematopoietic (an often dramatic drop in blood cells) leading to bleeding or anima and a suppressed immune system, gastrointestinal leading to fatigue, diarrhea, vomiting, nausea, fever, loss of appetite and abdominal pain and finally neurological and vascular injury which may lead to dizziness, seizures, and reduced cognitive abilities.
3. Methods of Priming Patients for Exposure to an Antineoplastic Agent

The compositions disclosed herein can also be used in prime healthy cell of a patient for exposure to an antineoplastic agent. Therefore, in some embodiments a subject's cells primed with the compositions disclosed herein can tolerate a higher dose of the antineoplastic agent compared to a subject that has not been given the composition. This method is particularly useful in patients undergoing cancer therapy, such as chemotherapy or radiation therapy as described above. In some embodiments, patients that could not otherwise tolerate the chemotherapeutic drug or radiation therapy, can tolerate the chemotherapeutic drug or radiation therapy with they are also administered the disclosed composition before, during, or after, exposure to the drug or radiation, or combination thereof. For example, in some embodiments the dosage of chemotherapeutic or radiation is a typical dosage that is known in the art, but which is prohibitive to some patients in the absence of the disclosed compositions due to toxic side effects. In some embodiments, the dosage of the chemotherapeutic or radiation is at the upper limit, or above the typical dosage that is known in the art due to toxic side effects, but can be tolerated by the patient in the presence of the disclosed compositions. The compositions can be used to increase a typical dosage of a chemotherapeutic drug or radiation therapy by between 101 and 500% inclusive.

B. Administration

The compositions provided herein may be administered in a physiologically acceptable carrier to a host. Preferred methods of administration include systemic or direct administration to a cell. The compositions can be administered to a cell or patient, as is generally known in the art for protein therapies. One embodiment provides a pharmaceutical composition includes a recombinant mitochondrial DNA-binding protein, for example a fusion protein. The fusion protein preferably contains polynucleotide-binding domain of a mitochondrial DNA-binding protein, a targeting domain, and a protein transduction domain and a pharmaceutically acceptable carrier or excipient. Preferably the polynucleotide-binding polypeptide includes TFAM or a fragment thereof capable of binding a polynucleotide. The composition typically includes an effective amount of the fusion protein to increase appetite, reduce weight loss, or a combination thereof in subject with a disease and disorders to be treated.

The compositions can be combined in admixture with a pharmaceutically acceptable carrier vehicle. Therapeutic formulations are prepared for storage by mixing the active ingredient having the desired degree of purity with optional physiologically acceptable carriers, excipients or stabilizers (Remington's Pharmaceutical Sciences 17th edition, Osol, A. Ed. (198)), in the form of lyophilized formulations or aqueous solutions. Acceptable carriers, excipients or stabilizers are nontoxic to recipients at the dosages and concentrations employed, and include buffers such as phosphate, citrate and other organic acids; antioxidants including ascorbic acid; low molecular weight (less than about 10 residues) polypeptides;

proteins, such as serum albumin, gelatin or immunoglobulins; hydrophilic polymers such as polyvinylpyrrolidone, amino acids such as glycine, glutamine, asparagine, arginine or lysine; monosaccharides, disaccharides and other carbohydrates including glucose, mannose, or dextrins; chelating agents such as EDTA; sugar alcohols such as mannitol or sorbitol; salt-forming counterions such as sodium; and/or nonionic surfactants such as Tween®, Pluronics® or PEG.

The compositions of the present disclosure can be administered parenterally. As used herein, “parenteral administration” is characterized by administering a pharmaceutical composition through a physical breach of a subject's tissue. Parenteral administration includes administering by injection, through a surgical incision, or through a tissue-penetrating non-surgical wound, and the like. In particular, parenteral administration includes subcutaneous, intraperitoneal, intravenous, intraarterial, intramuscular, intrasternal injection, and kidney dialytic infusion techniques.

Parenteral formulations can include the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Parenteral administration formulations include suspensions, solutions, emulsions in oily or aqueous vehicles, pastes, reconsitutable dry (i.e. powder or granular) formulations, and implantable sustained-release or biodegradable formulations. Such formulations may also include one or more additional ingredients including suspending, stabilizing, or dispersing agents. Parenteral formulations may be prepared, packaged, or sold in the form of a sterile injectable aqueous or oily suspension or solution. Parenteral formulations may also include dispersing agents, wetting agents, or suspending agents described herein. Methods for preparing these types of formulations are known. Sterile injectable formulations may be prepared using non-toxic parenterally-acceptable diluents or solvents, such as water, 1,3-butane diol, Ringer's solution, isotonic sodium chloride solution, and fixed oils such as synthetic monoglycerides or diglycerides. Other parentally-administrable formulations include microcrystalline forms, liposomal preparations, and biodegradable polymer systems. Compositions for sustained release or implantation may include pharmaceutically acceptable polymeric or hydrophobic materials such as emulsions, ion exchange resins, sparingly soluble polymers, and sparingly soluble salts.

Pharmaceutical compositions may be prepared, packaged, or sold in a buccal formulation. Such formulations may be in the form of tablets, powders, aerosols, atomized solutions, suspensions, or lozenges made using known methods, and may contain from about 0.1% to about 20% (w/w) active ingredient with the balance of the formulation containing an orally dissolvable or degradable composition and/or one or more additional ingredients as described herein. Preferably, powdered or aerosolized formulations have an average particle or droplet size ranging from about 0.1 nanometers to about 200 nanometers when dispersed.

The composition can include one or more additional ingredients. As used herein, “additional ingredients” include: excipients, surface active agents, dispersing agents, inert diluents, granulating agents, disintegrating agents, binding agents, lubricating agents, sweetening agents, flavoring agents, coloring agents, preservatives, physiologically degradable compositions (e.g., gelatin), aqueous vehicles, aqueous solvents, oily vehicles and oily solvents, suspending agents, dispersing agents, wetting agents, emulsifying agents, demulcents, buffers, salts, thickening agents, fillers, emulsifying agents, antioxidants, antibiotics, antifungal agents, stabilizing agents, and pharmaceutically acceptable polymeric or hydrophobic materials. Other additional ingredients which may be included in the pharmaceutical compositions are known. Suitable additional ingredients are described in Remington's Pharmaceutical Sciences, 17^{th} ed. Mack Publishing Co., Genaro, ed., Easton, Pa. (1985).

Dosages and desired concentrations of the polynucleotide-binding polypeptide disclosed herein in pharmaceutical compositions of the present disclosure may vary depending on the particular use envisioned. The determination of the appropriate dosage or route of administration is well within the skill of an ordinary physician. Animal experiments provide reliable guidance for the determination of effective doses for human therapy. Interspecies scaling of effective doses can be performed following the principles laid down by Mordenti, J. and Chappell, W. “The use of interspecies scaling in toxicokinetics” In Toxicokinetics and New Drug Development, Yacobi et al., Eds., Pergamon Press, New York 1989, pp. 42-96.

The composition can be administered intravenously in a wide dosing range from about 0.01 milligram per kilo body weight (mg/kg) to about 1.0 mg/kg, depending on patient's age and physical state, as well as dosing regimen and schedule.

In some embodiments the composition is lyophilized in 20 mM glutamate, 10 mg/mL trehalose, 30 mg/mL mannitol, pH 4.5 and reconstituted in sterile water prior to use. In another embodiment the composition is lyophilized in 20 mM histidine, 10 mg/mL trehalose, 30 mg/mL mannitol, pH 6.5 and reconstituted in sterile water prior to use. In yet another embodiment, the composition is dissolved in 20 mM histidine, 150 mM NaCl pH 6.5 and kept frozen prior to use.

### III. Compositions

Compositions for treating one or more symptoms of sides effects associated with the treatment of hyperproliferative disorders or exposure to radiation. In some embodiments, the disclosed compositions cause an increase in mitochondrial number, can increase in mitochondrial respiration relative to a control, or both. The composition typically includes an effective amount of a mitochondrial DNA-binding polypeptide. Examples of a mitochondrial DNA-binding polypeptides include, but are not limited to, mitochondrial transcription factors such as transcription factor A, mitochondrial (TFAM) having GenBank Accession No. mitochondrial NM_{—}003201; transcription factor B1, mitochondrial (TFB1M) having GenBank Accession No. AF151833; transcription factor B2, mitochondrial (TFB2M) having GenBank Accession No. AK026835; Polymerase (RNA) Mitochondrial (DNA directed) (POLRMT) having GenBank Accession No. NM_{—}005035; and functional fragments, variants, and fusion polypeptides thereof.

In preferred embodiments the composition includes a recombinant fusion protein including a polynucleotide-binding polypeptide, a protein transduction domain, and optionally one or more targeting signals. In some embodiments, the disclosed compositions cause an increase in mitochondrial number, an increase in mitochondrial respiration, an increase mitochondrial Electron Transport Chain (ETC) activity, increased oxidative phosphorylation, increased oxygen consumption, increased ATP production, or combinations thereof relative to a control. In preferred embodiments the composition reduces oxidative stress.

Exemplary fusion proteins containing a mitochondrial transcription factor polypeptide are disclosed in U.S. Pat. Nos. 8,039,587, 8,062,891, 8,133,733, and U.S. Published Application Nos. 2009/0123468, 2009/0208478, and 2006/0211647 all of which are specifically incorporated by reference herein in their entireties.

A. Polypeptides
1. Polynucleotide Binding Domain

The compositions for treating one or more symptoms of sides effects associated with cancer treatments provided herein include an effective amount of a mitochondrial DNA-binding polypeptide optionally having a PTD and optionally having one or more targeting signals or domains. In certain embodiments, the mitochondrial DNA-binding polypeptide is a polypeptide known to bind or package a mtDNA. Preferably, the mitochondrial DNA-binding polypeptide is a recombinant polypeptide. The recombinant polypeptide can be used as a therapeutic agent either alone or in combination with a polynucleotide, or any other active agent. In preferred embodiments the polynucleotide-binding domain includes mature TFAM, a functional fragment of TFAM, or a variant thereof. In certain embodiments, the polynucleotide-binding polypeptide includes at least a portion of a member of the high mobility group (HMG) of proteins effective to bind a polynucleotide, for example an HMG box domain.

“Mature TFAM” refers to TFAM after it has been post-translationally modified and is in the form that is active in the mitochondrion. For example, a mature TFAM is one in which the endogenous mitochondrial signal sequence has been cleaved.
a. Transcription Factor A, Mitochondria (TFAM)

One embodiment provides a non-histone polynucleotide-binding polypeptide, for example mitochondrial transcription factor A (TFAM) polypeptide, for functional fragment, or a variant thereof. Variant TFAM can have 80%, 85%, 90%, 95%, 99% or greater sequence identity with a reference TFAM, for example naturally occurring TFAM having GenBank Accession No. NM_{—}003201. In certain embodiments, the variant TFAM has 80%, 85%, 90%, 95%, 99% or greater sequence identity with a reference TFAM. In certain embodiments, the variant TFAM has 80%, 85%, 90%, 95%, 99% or greater sequence identity over the full-length of mature human TFAM.

TFAM is a member of the high mobility group (HMG) of proteins having two HMG-box domains. TFAM as well as other HMG proteins bind, wrap, bend, and unwind DNA. Thus, embodiments of the present disclosure include polynucleotide binding polypeptides including one or more polynucleotide binding regions of the HMG family of proteins, and optionally induce a structural change in the polynucleotide when the polypeptide binds or becomes associated with the polynucleotide.

In some embodiment, the polynucleotide-binding polypeptide is full-length TFAM polypeptide, or variant therefore. For example, a preferred TFAM polypeptide has at least 80, 85, 90, 95, 99, or 100 percent sequence identity to the full-length TFAM precursor

(SEQ ID NO: 1)
MAFLRSMWGV LSALGRSGAE LCTGCGSRLR SPFSFVYLPR
WFSSVLASCP KKPVSSYLRF SKEQLPIFKA QNPDAKTTEL
IRRIAQRWRE LPDSKKKIYQ DAYRAEWQVY KEEISRFKEQ
LTPSQIMSLE KEIMDKHLKR KAMTKKKELT LLGKPKRPRS
AYNVYVAERF QEAKGDSPQE KLKTVKENWK NLSDSEKELY
IQHAKEDETR YHNEMKSWEE QMIEVGRKDL LRRTIKKQRK
YGAEEC.

Many nuclear encoded mitochondrial proteins destined for the mitochondrial matrix are translated as a “preprotein.” The preprotein sequence includes a signal peptide as known as an “amino-terminal signal”, or a “presequence” that facilitates translocation from the cytosol through the mitochondrial translocation machinery in the outer membrane called the

Tom complex (Translocator outer membrane) as well as the machinery in the inner membrane called the Tim complex (Translocator Inner Membrane). Once the preprotein enters the inner mitochondrial matrix, the signal sequence is cleaved by a protease such as MPP. A mitochondrial protein with the signal sequence cleaved or removed can be referred to as a “mature” protein. Therefore, in some embodiments, the polynucleotide-binding polypeptide is a mature TFAM polypeptide, or variant thereof. For example, in some embodiments, the cleavable mitochondrial targeting sequence of a TFAM preprotein is amino acid residue 1 of SEQ ID NO:1 to amino acid residue 42 of SEQ ID NO:1, MAFLRSMWGV LSALGRSGAE LCTGCGSRLR SPFSFVYLPR WF

(SEQ ID NO: 2). In certain embodiments, a preferred TFAM polypeptide has at least 80, 85, 90, 95, 99, or 100 percent sequence identity to the mature TFAM sequence

(SEQ ID NO: 3)
SSVLASCPKK PVSSYLRFSK EQLPIFKAQN PDAKTTELIR
RIAQRWRELP DSKKKIYQDA YRAEWQVYKE EISRFKEQLT
PSQIMSLEKE IMDKHLKRKA MTKKKELTLL GKPKRPRSAY
NVYVAERFQE AKGDSPQEKL KTVKENWKNL SDSEKELYIQ
HAKEDETRYH NEMKSWEEQM IEVGRKDLLR RTIKKQRKYG AEEC.

In some embodiments, the polynucleotide-binding polypeptide is functional fragment of TFAM, or variant therefore. A functional fragment of TFAM as used herein is a fragment of full-length TFAM that is when administered to a patient reduces, inhibits or alleviates, one or more symptoms or sides effects associated with physical insult caused by or chemotherapy or high levels of radiation compared to a control. Functional fragments can be effective when administered alone, or can be effective when administered in combination with a polynucleotide. Functional fragments of TFAM can include, but are not limited to, a fragment of full-length TFAM sufficient to bind non-specifically to a polynucleotide, a fragment of full-length TFAM sufficient to bind specifically to the mtDNA light strand promoter (LSP), the mtDNA heavy strand promoter 1 (HSP1), the mtDNA heavy stand promoter 2 (HSP2), or combinations thereof, a fragment of full-length TFAM sufficient to induce mitochondrial transcription, a fragment of full-length TFAM sufficient to induce oxidative phosphorylation, a fragment of full-length TFAM sufficient to induce mitochondrial biogenesis, and combinations thereof.

From N-terminus to C-terminus, mature TFAM includes four domains, a first HMG box (also referred to herein as HMG box 1), followed by a linker region (also referred to herein as linker), followed by a second HMG box (also referred to herein as HMG box 2), followed by a C-terminal tail. Functional fragments of TFAM typically include one or more domains of mature TFAM, or a variant thereof. For example, in some embodiments, the functional fragment includes one or more HMG box 1 domains of TFAM, one or more linker domains of TFAM, one or more HMG box 2 domains of TFAM, one or more C-terminal tail domains of TFAM, or combinations thereof. The domains can be arranged in the same orientation of the domains of endogenous TFAM, or they can be rearranged so they are in a different order or orientation than the domains found in endogenous TFAM protein. In certain embodiments the functional fragment includes a first HMG box domain, and second HMG box domain linked to the first HMG box domain with a linker, typically a peptide linker. The linker can be the endogenous linker domain of TFAM, or a heterologous linker that allows the first and the second HMG box domains to maintain their functional activity. Deletion studies characterizing the activity of different domains and hybrid constructs of TFAM are known in the art and characterized for example in Dairaghi, et al., J. Mol. Biol., 249:11-28 (1995), Matsushima, et al., J. Biol. Chem., 278(33):31149-31158 (2003), and Gangeloff, et al., Nucl. Acid. Res., 37(10):3153-3164 (2009), all of which are specifically incorporated by reference herein in their entireties.

In certain embodiments a functional fragment is one or more domains of TFAM according to SEQ ID NO: 3. For example, an HMG box 1 of TFAM can be a polypeptide including the sequence from amino acid residue 1, 2, 3, 4, 5, 6, 7, 8, 9, or 10 of SEQ ID NO: 3 to amino acid residue 70, 71, 72, 73, 74, 75, 76, 77, 78, 79, 80, 81, 82, 83, 84, 85, 86, 87, 88, 89, 90, 91, 92, 93, 94, or 95 of SEQ ID NO: 3, or a variant thereof with 80, 85, 90, 95, 99, or greater than 99 percent sequence identity to the corresponding fragment of SEQ ID NO: 3.

A linker region of TFAM can be a polypeptide including the sequence from amino acid residue 70, 71, 72, 73, 74, 75, 76, 77, 78, 79, 80, 81, 82, 83, 84, or 85 of SEQ ID NO: 3 to amino acid residue 105, 106, 107, 108, 109, 110, 111, 112, 113, 114, or 115 of SEQ ID NO: 3, or a variant thereof with 80, 85, 90, 95, 99, or greater than 99 percent sequence identity to the corresponding fragment of SEQ ID NO: 3.

An HMG box 2 of TFAM can be a polypeptide including the sequence from amino acid residue 96, 97, 98, 99, 100, 101, 102, 103, 104, 105, 106, 107, 108, 109, 110, 111, 112, 113, 114, or 115 of SEQ ID NO: 3 to amino acid residue 172, 173, 174, 175, 176, 177, 178, 179, 180, 181, 182, 183, 184, 185, 186, or 187 of SEQ ID NO: 3, or a variant thereof with 80, 85, 90, 95, 99, or greater than 99 percent sequence identity to the corresponding fragment of SEQ ID NO: 3.

A C-terminal tail of TFAM can be a polypeptide including the sequence from amino acid residue 172, 173, 174, 175, 176, 177, 178, 179, 180, 181, 182, 183, 184, 185, 186, or 187 of SEQ ID NO: 3 to amino acid residue 194, 195, 196, 197, 198, 199, 200, 201, 202, 203, or 204 of SEQ ID NO: 3, or a variant thereof with 80, 85, 90, 95, 99, or greater than 99 percent sequence identity to the corresponding fragment of SEQ ID NO: 3.

Variants of TFAM and functional fragments of TFAM are also provided. Typically, the variants of TFAM and function fragments of TFAM include one or more conservative amino acid substitutions relative to the corresponding reference sequence, for example SEQ ID NO:3, or a fragment thereof. One embodiment provides a TFAM polypeptide having one or more serine residues at positions 1, 2 and 13 SEQ ID NO:3 substituted with an alanine or threonine residue. A preferred embodiment provides a TFAM polypeptide having serine 13 of SEQ ID NO:3 substituted for an alanine or threonine. The variant TFAM polypeptides have improved mtDNA binding in the presence of glucose or elevated glucose levels.

Selected model organisms that have TFAM sequences that are useful in the compositions and methods disclosed herein include, but are not limited to those disclosed in Table 1:

TABLE 1
Organism, Protein And Percent Identity
And Length Of Aligned Region

| | | |
|---|---|---|
| H. sapiens | sp: Q00059 - MTT1_HUMAN | 100%/246 aa |
| | Transcription factor 1, mitochondrial | (see ProtEST) |
| | precursor (MTTF1) | |
| M. musculus | ref: NP_033386.1 - transcription | 63%/237 aa |
| | factor A, mitochondrial | (see ProtEST) |
| | [Mus musculus] | |
| R. norvegicus: | ref: NP_112616.1 - transcription | 64%/237 aa |
| | factor A, mitochondrial | (see ProtEST) |
| | [Rattus norvegicus] | |
| A. thaliana | ref: NP_192846.1 - 98b like protein | 27%/189 aa |
| | [Arabidopsis thaliana] | (see ProtEST) |
| C. elegans | ref: NP_501245.1 - F45E4.9.p | 27%/189 aa |
| | [Caenorhabditis elegans] | (see ProtEST) |
| D. melanogaster: | ref: NP_524415.1 - mitochondrial | 34%/183 aa |
| | transcription factor A | (see ProtEST) |
| | [Drosophila melanogaster] | |

b. Transcription Factor B1, Mitochondrial (TFB1M)

The polynucleotide-binding polypeptide can be transcription factor B1, mitochondrial (TFB1M). A preferred TFB1M has GenBank Accession No. AF151833. TFB1 is part of the complex involved in mitochondrial transcription. The process of transcription initiation in mitochondria involves three types of proteins: the mitochondrial RNA polymerase (POLRMT), mitochondrial transcription factor A (TFAM), and mitochondrial transcription factors B1 and B2 (TFB1M, TFB2M). POLRMT, TFAM, and TFB 1M or TFB2M assemble at the mitochondrial promoters and begin transcription. TFB1M has about 1/10 the transcriptional activity of TFB2M, and both TFBs are also related to rRNA methyltransferases and TFB1M can bind S-adenosylmethionine and methylate mitochondrial 12S rRNA. Additionally, TFB1M and TFB2M can bind single-stranded nucleic acids.

A preferred TFB1M polypeptide has at least 80, 85, 90, 95, 99, or 100 percent sequence identity to

(SEQ ID NO: 4)
MAASGKLSTC RLPPLPTIRE IIKLLRLQAA NELSQNFLLD
LRLTDKIVRK AGNLTNAYVY EVGPGPGGIT RSILNADVAE
LLVVEKDTRF IPGLQMLSDA APGKLRIVHG DVLTFKVEKA
FSESLKRPWE DDPPNVHIIG NLPFSVSTPL IIKWLENISC
RDGPFVYGRT QMTLTFQKEV AERLAANTGS KQRSRLSVMA
QYLCNVRHIF TIPGQAFVPK PEVDVGVVHF TPLIQPKIEQ
PFKLVEKVVQ NVFQFRRKYC HRGLRMLFPE AQRLESTGRL
LELADIDPTL RPRQLSISHF KSLCDVYRKM CDEDPQLFAY
NFREELKRRK SKNEEKEEDD AENYRL.

c. Transcription Factor B2, Mitochondrial (TFB2M)

In still another embodiment, the polynucleotide-binding polypeptide includes TFB2M. In a preferred embodiment the TFB2M polypeptide has GenBank Accession No. AK026835. TFB2M also possesses a Rossmann-fold making it part of the NAD-binding protein family. TFB2M levels modulate mtDNA copy number and levels of mitochondrial transcripts as would be expected of a mitochondrial transcription factor. It is appreciated by those skilled in the art that increased activity of mitochondria causes an increase in mitochondrial biogenesis.

A preferred TFB2M polypeptide has at least 80, 85, 90, 95, 99, or 100 percent sequence identity to

(SEQ ID NO: 5)
MWIPVVGLPR RLRLSALAGA GRFCILGSEA ATRKHLPARN
HCGLSDSSPQ LWPEPDFRNP PRKASKASLD FKRYVTDRRL
AETLAQIYLG KPSRPPHLLL ECNPGPGILT QALLEAGAKV
VALESDKTFI PHLESLGKNL DGKLRVIHCD FFKLDPRSGG
VIKPPAMSSR GLFKNLGIEA VPWTADIPLK VVGMFPSRGE
KRALWKLAYD LYSCTSIYKF GRIEVNMFIG EKEFQKLMAD
PGNPDLYHVL SVIWQLACEI KVLHMEPWSS FDIYTRKGPL
ENPKRRELLD QLQQKLYLIQ MIPRQNLFTK NLTPMNYNIF
FHLLKHCFGR RSATVIDHLR SLTPLDARDI LMQIGKQEDE
KVVNMHPQDF KTLFETIERS KDCAYKWLYD ETLEDR.

d. Polymerase (RNA) Mitochondrial (DNA directed) (POLRMT)

Still another polynucleotide-binding polypeptide that can be used to modulate mitochondrial biological activity is POLRMT. In a preferred embodiment, the POLRMT polypeptide has GenBank Accession No. NM_{—}005035. POLRMT is a mitochondrial RNA polymerase similar in structure to phage RNA polymerases. Unlike phage polymerases, POLRMT contains two pentatricopeptide repeat (PPR) domains involved in regulating mitochondrial transcripts. It is appreciated by those skilled in the art that deletion of regulatory domains enables constitutive function.

A preferred POLRMT polypeptide has at least 80, 85, 90, 95, 99, or 100 percent sequence identity to

(SEQ ID NO: 39)
MSALCWGRGA AGLKRALRPC GRPGLPGKEG TAGGVCGPRR
SSSASPQEQD QDRRKDWGHV ELLEVLQARV RQLQAESVSE
VVVNRVDVAR LPECGSGDGS LQPPRKVQMG AKDATPVPCG
RWAKILEKDK RTQQMRMQRL KAKLQMPFQS GEFKALTRRL
QVEPRLLSKQ MAGCLEDCTR QAPESPWEEQ LARLLQEAPG
KLSLDVEQAP SGQHSQAQLS GQQQRLLAFF KCCLLTDQLP
LAHHLLVVHH GQRQKRKLLT LDMYNAVMLG WARQGAFKEL
VYVLFMVKDA GLTPDLLSYA AALQCMGRQD QDAGTIERCL
EQMSQEGLKL QALFTAVLLS EEDRATVLKA VHKVKPTFSL
PPQLPPPVNT SKLLRDVYAK DGRVSYPKLH LPLKTLQCLF
EKQLHMELAS RVCVVSVEKP TLPSKEVKHA RKTLKTLRDQ
WEKALCRALR ETKNRLEREV YEGRFSLYPF LCLLDEREVV
RMLLQVLQAL PAQGESFTTL ARELSARTFS RHVVQRQRVS
GQVQALQNHY RKYLCLLASD AEVPEPCLPR QYWEELGAPE
ALREQPWPLP VQMELGKLLA EMLVQATQMP CSLDKPHRSS
RLVPVLYHVY SFRNVQQIGI LKPHPAYVQL LEKAAEPTLT
FEAVDVPMLC PPLPWTSPHS GAFLLSPTKL MRTVEGATQH
QELLETCPPT ALHGALDALT QLGNCAWRVN GRVLDLVLQL
FQAKGCPQLG VPAPPSEAPQ PPEAHLPHSA APARKAELRR
ELAHCQKVAR EMHSLRAEAL YRLSLAQHLR DRVFWLPHNM
DFRGRTYPCP PHFNHLGSDV ARALLEFAQG RPLGPHGLDW
LKIHLVNLTG LKKREPLRKR LAFAEEVMDD ILDSADQPLT
GRKWWMGAEE PWQTLACCME VANAVRASDP AAYVSHLPVH
QDGSCNGLQH YAALGRDSVG AASVNLEPSD VPQDVYSGVA
AQVEVFRRQD AQRGMRVAQV LEGFITRKVV KQTVMTVVYG
VTRYGGRLQI EKRLRELSDF PQEFVWEASH YLVRQVFKSL
QEMFSGTRAI QHWLTESARL ISHMGSVVEW VTPLGVPVIQ
PYRLDSKVKQ IGGGIQSITY THNGDISRKP NTRKQKNGFP
PNFIHSLDSS HMMLTALHCY RKGLTFVSVH DCYWTHAADV
SVMNQVCREQ FVRLHSEPIL QDLSRFLVKR FCSEPQKILE
ASQLKETLQA VPKPGAFDLE QVKRSTYFFS.

e. HMG Domain

In some embodiments, the polynucleotide-binding polypeptide is a non-TFAM HMG domain. Generally, the HMG domain includes a global fold of three helices stabilized in an ‘L-shaped’ configuration by two hydrophobic cores. The high mobility group chromosomal proteins HMG1 or HMG2, which are common to all eukaryotes, bind DNA in a non-sequence-specific fashion, for example to promote chromatin function and gene regulation. They can interact directly with nucleosomes and are believed to be modulators of chromatin structure. They are also important in activating a number of regulators of gene expression, including p53, Hox transcription factors and steroid hormone receptors, by increasing their affinity for DNA. HMG proteins include HMG-1/2, HMG-I(Y) and HMG-14/17.

The HMG-1/2-box proteins can be further distinguished into three subfamilies according to the number of HMG domains present in the protein, their specific of sequence recognition and their evolutionary relationship. The first group contains chromosomal proteins bound to DNA with no sequence specificity (class I, HMG1 and HMG2), the second contains ribosomal and mitochondrial transcription factors which show sequence specificity in the presence of another associating factor when bound with DNA (class II, yeast ARS binding protein ABF-2, UBF and mitochondrial transcription factor mtTF-1), and the third contains gene-specific transcription factors which show sequence specific DNA binding (class III, lymphoid enhancer-binding factors LEF-1 and TCF-1; the mammalian sex-determining factor SRY, and the closely related SOX proteins; and the fungal regulatory proteins Mat-MC, Mat-al, Stell and Roxl). The HMG1/2-box DNA binding domain is about 75 to about 80 amino acids and contains highly conserved proline, aromatic and basic residues. Common properties of HMG domain proteins include interaction with the minor groove of the DNA helix, binding to irregular DNA structure, and the capacity to modulate DNA structure by bending.

SOX (SRY-type HMG box) proteins have critical functions in a number of developmental processes, including sex determination, skeleton formation, pre-B and T cell development and neural induction. SOX9 plays a direct role during chondrogenesis by binding and activating the chondrocyte-spacific enhancer of the Col2a1 gene. Loss of SOX9 gene function leads to the genetic condition known as Campomelic Dysplsia (CD), a form of dwarfism characterized by extreme skeletal malformation, and one in which three-quarters of XY individual are either intersexes or exhibit male to female sex reversal. There are more than 20 members cloned in SOX family. All of which contain an HMG domain, which can bind specifically to the double strand DNA motif and shares >50% identify with the HMG domain of SRY, the human testis-determining factor. The preferred DNA-binding site of SOX9 have been defined to be AGAACAATGG (SEQ ID NO: 6), which contains the SOX core-binding element (SCBE), AACAAT, flanking 5′ AG and 3′ GG nucleotides enhance binding by SOX9.

In one embodiment, the recombinant polynucleotide-binding polypeptide has at least one HMG box domain, generally at least two, more particularly 2-5 HMG box domains. The HMG box domain can bind to an AT rich DNA sequence, for example, using a large surface on the concave face of the protein, to bind the minor groove of the DNA. This binding bends the DNA helix axis away from the site of contact. The first and second helices contact the DNA, their N-termini fitting into the minor groove whereas helix 3 is primarily exposed to solvent. Partial intercalation of aliphatic and aromatic residues in helix 2 occurs in the minor groove.

In other embodiments, the polynucleotide-binding polypeptide can have at least one polynucleotide binding domain, typically two or more polynucleotide binding domains. The polynucleotide binding domains can be the same or different. For example, the polynucleotide-binding polypeptide can include at least one HMG box in combination with one or more DNA binding domains selected from the group consisting of an HMG box, homeodomain and POU domain; zinc finger domain such as C₂H₂ and C₂C₂; amphipathic helix domain such as leucine zipper and helix-loop-helix domains; and histone folds. The polynucleotide binding domain can be specific for a specific polynucleotide sequence, or preferably non-specifically binds to a polynucleotide. Alternatively, the polynucleotide-binding polypeptide can have more a combination of at least one polynucleotide binding domain that binds in a sequence specific manner and at least one polynucleotide binding-domain that binds DNA non-specifically.
f. Helix-Turn-Helix

Certain embodiments provide polynucleotide-binding polypeptides having a helix-turn-helix motif or at least a polynucleotide binding region of a helix-turn-helix protein. Helix-turn-helix proteins have a similar structure to bacterial regulatory proteins such as the 1 repressor and cro proteins, the lac repressor and so on which bind as dimers and their binding sites are palindromic. They contain 3 helical regions separated by short turns which is why they are called helix-turn-helix proteins. One protein helix (helix 3) in each subunit of the dimer occupies the major groove of two successive turns of the DNA helix. Thus, in another embodiment, the disclosed polynucleotide-binding polypeptides can form dimers or other multi-component complexes, and have 1 to 3 helices.
g. Homeodomain

In yet another embodiment, the polynucleotide-binding polypeptide includes a homeodomain or a portion of a homeodomain protein. Homeodomain proteins bind to a sequence of 180 base pairs initially identified in a group of genes called homeotic genes. Accordingly, the sequence was called the homeobox. The 180 by corresponds to 60 amino acids in the corresponding protein. This protein domain is called the homeodomain. Homeodomain-containing proteins have since been identified in a wide range of organisms including vertebrates and plants. The homeodomain shows a high degree of sequence conservation. The homeodomain contains 4 a helical regions. Helices II and III are connected by 3 amino acids comprising a turn. This region has a very similar structure to helices II and III of bacterial DNA binding proteins.
h. Zinc Finger

Yet another embodiment provides a modified polynucleotide-binding polypeptide having a zinc finger domain or at least a portion of a zinc finger protein. Zinc finger proteins have a domain with the general structure: Phe (sometimes Tyr)—Cys—2 to 4 amino acids—Cys—3 amino acids—Phe (sometimes Tyr)—5 amino acids—Leu—2 amino acids—His—3 amino acids—His. The phenylalanine or tyrosine residues which occur at invariant positions are required for DNA binding. Similar sequences have been found in a range of other DNA binding proteins though the number of fingers varies. For example, the SP1 transcription factor which binds to the GC box found in the promoter proximal region of a number of genes has 3 fingers. This type of zinc finger which has 2 cysteines and 2 histidines is called a C₂H₂ zinc finger.

Another type of zinc finger which binds zinc between 2 pairs of cysteines has been found in a range of DNA binding proteins. The general structure of this type of zinc finger is: Cys—2 amino acids—Cys—13 amino acids—Cys—2 amino acids—Cys. This is called a C₂C₂ zinc finger. It is found in a group of proteins known as the steroid receptor superfamily, each of which has 2 C₂C₂ zinc fingers.
i. Leucine Zipper

Another embodiment provides a modified polynucleotide-binding polypeptide having a leucine zipper or at least a portion of a leucine zipper protein. The first leucine zipper protein was identified from extracts of liver cells, and it was called C/EBP because it is an enhancer binding protein and it was originally thought to bind to the CAAT promoter proximal sequence. C/EBP will only bind to DNA as a dimer. The region of the protein where the two monomers join to make the dimer is called the dimerization domain. This lies towards the C-terminal end of the protein. When the amino acid sequence was examined it was found that a leucine residue occurs every seventh amino acid over a stretch of 35 amino acids. If this region were to form an a helix then all of these leucines would align on one face of the helix.

Because leucine has a hydrophobic side chain, one face of the helix is very hydrophobic. The opposite face has amino acids with charged side chains which are hydrophilic. The combination of hydrophobic and hydrophilic characteristics gives the molecule is amphipathic moniker. Adjacent to the leucine zipper region is a region of 20-30 amino acids which is rich in the basic (positively charged) amino acids lysine and arginine. This is the DNA binding domain—often referred to as the bZIP domain—the basic region of the leucine zipper. C/EBP is thought to bind to DNA by these bZIP regions wrapping round the DNA helix

The leucine zipper—bZIP structure has been found in a range of other proteins including the products of the jun and fos oncogenes. Whereas C/EBP binds to DNA as a homodimer of identical subunits, fos cannot form homodimers at all and jun/jun homodimers tend to be unstable. However fos/jun heterodimers are much more stable. These fos/jun heterodimers correspond to a general transcription factor called AP 1 which binds to a variety of promoters and enhancers and activates transcription. The consensus AP1 binding site is TGACTCA which is palindromic.
j. Helix-Loop-Helix

Another embodiment provides a modified polynucleotide-binding polypeptide having helix-loop-helix domain or a polynucleotide binding portion of a helix-loop-helix protein. Helix-loop-helix proteins are similar to leucine zippers in that they form dimers via amphipathic helices. They were first discovered as a class of proteins when a region of similarity was noticed between two enhancer binding proteins called E47 and E12. This conserved region has the potential to form two amphipathic separated by a loop hence helix-loop-helix. Next to the dimerization domain is a DNA binding domain, again rich in basic amino acids and referred to as the bHLH domain. These structures are also found in a number of genes required for development of the Drosophila nervous system—the Achaete-scute complex, and in a protein called MyoD which is required for mammalian muscle differentiation.
k. Histone Fold

In still another embodiment, the modified polynucleotide-binding polypeptide includes a histone polypeptide, a fragment of a histone polypeptide, or at least one histone fold. Histone folds exist in histone polypeptides monomers assembled into dimers. Histone polypeptides include H2A, H2B, H3, and H4 which can form heterodimers H2A-2B and H3-H4. It will be appreciated that histone-like polypeptides can also be used in the disclosed compositions and methods. Histone-like polypeptides include, but are not limited to, HMf or the histone from Methanothermous fervidus, other archaeal histones known in the art, and histone-fold containing polypeptides such as MJ1647, CBF, TAFII or transcription factor IID, SPT3, and Dr1-DRAP (Sanderman, K., et al., Cell. Mol. Life Sci. 54:1350-1364 (1998), which is specifically incorporated by reference herein in its entirety).
2. Protein Transduction Domain

In some embodiments, the polynucleotide-binding polypeptide is fusion protein modified to include a protein transduction domain (PTD). As used herein, a “protein transduction domain” or PTD refers to a polypeptide, polynucleotide, carbohydrate, organic or inorganic compound that facilitates traversing a lipid bilayer, micelle, cell membrane, organelle membrane, or vesicle membrane. A PTD attached to another molecule facilitates the molecule traversing membranes, for example going from extracellular space to intracellular space, or cytosol to within an organelle.

In preferred embodiments, the protein transduction domain is a polypeptide. A protein transduction domain can be a polypeptide including positively charged amino acids. Thus, some embodiments include PTDs that are cationic or amphipathic. Protein transduction domains (PTD), also known as a cell penetrating peptides (CPP), are typically polypeptides including positively charged amino acids. PTDs are known in the art, and include but are not limited to small regions of proteins that are able to cross a cell membrane in a receptor-independent mechanism (Kabouridis, P., Trends in Biotechnology (11):498-503 (2003)). Although several PTDs have been documented, the two most commonly employed PTDs are derived from TAT (Frankel and Pabo, Cell, 55(6):1189-93(1988)) protein of HIV and Antennapedia transcription factor from Drosophila, whose PTD is known as Penetratin (Derossi et al., J Biol Chem., 269(14):10444-50 (1994)). Exemplary protein transduction domains include polypeptides with 11 Arginine residues, or positively charged polypeptides or polynucleotides having 8-15 residues, preferably 9-11 residues.

The Antennapedia homeodomain is 68 amino acid residues long and contains four alpha helices. Penetratin is an active domain of this protein which consists of a 16 amino acid sequence derived from the third helix of Antennapedia. TAT protein consists of 86 amino acids and is involved in the replication of HIV-1. The TAT PTD consists of an 11 amino acid sequence domain (residues 47 to 57; YGRKKRRQRR R (SEQ ID NO:7)) of the parent protein that appears to be critical for uptake. Additionally, the basic domain Tat(49-57) or RKKRRQRRR (SEQ ID NO:8) has been shown to be a PTD. In the current literature TAT has been favored for fusion to proteins of interest for cellular import. Several modifications to TAT, including substitutions of Glutatmine to Alanine, i.e., Q→A, have demonstrated an increase in cellular uptake anywhere from 90% (Wender et al., Proc Natl Acad Sci USA., 97(24):13003-8 (2000)) to up to 33 fold in mammalian cells. (Ho et al., Cancer Res., 61(2):474-7 (2001)).

The most efficient uptake of modified proteins was revealed by mutagenesis experiments of TAT-PTD, showing that an 11 arginine stretch was several orders of magnitude more efficient as an intercellular delivery vehicle. Therefore, PTDs can include a sequence of multiple arginine residues, referred to herein as poly-arginine or poly-ARG. In some embodiments the sequence of arginine residues is consecutive. In some embodiments the sequence of arginine residues is non-consecutive. A poly-ARG can include at least 7 arginine residues, more preferably at least 8 arginine residues, most preferably at least 11 arginine residues. In some embodiments, the poly-ARG includes between 7 and 15 arginine residues, more preferably between 8 and 15 arginine residues. In some embodiments the poly-ARG includes between 7 and 15, more preferably between 8 and 15 consecutive arginine residues. An example of a poly-ARG is RRRRRRR (SEQ ID NO:9). Additional exemplary PTDs include but are not limited to; RRQRRTSKLM KR (SEQ ID NO:10); GWTLNSAGYL LGKINLKALA ALAKKIL (SEQ ID NO:11); WEAKLAKALA KALAKHLAKA LAKALKCEA (SEQ ID NO:12); and RQIKIWFQNR RMKWKK (SEQ ID NO:13).

Without being bound by theory, it is believed that following an initial ionic cell-surface interaction, some polypeptides containing a protein transduction domain are rapidly internalized by cells via lipid raft-dependent macropinocytosis. For example, transduction of a TAT-fusion protein was found to be independent of interleukin-2 receptor/raft-, caveolar- and clathrin-mediated endocytosis and phagocytosis (Wadia, et al., Nature Medicine, 10:310-315 (2004), and Barka, et al., J. Histochem. Cytochem., 48(11):1453-60 (2000)). Therefore, in some embodiments the polynucleotide-binding polypeptide includes an endosomal escape sequence that enhances escape of the polypeptide-binding protein from macropinosomes. The some embodiments the endosomal escape sequence is part of, or consecutive with, the protein transduction domain. In some embodiments, the endosomal escape sequence is non-consecutive with the protein transduction domain. In some embodiments the endosomal escape sequence includes a portion of the hemagglutinin peptide from influenza (HA). One example of an endosomal escape sequence includes GDIMGEWG NEIFGAIAGF LG (SEQ ID NO:14).

In one embodiment a protein transduction domain including an endosomal escape sequence includes the amino acid sequence RRRRRRRRRR RGEGDIMGEW GNEIFGAIAG FLGGE (SEQ ID NO:15).

3. Targeting Signal or Domain

In some embodiments the polynucleotide-binding polypeptide is modified to include one or more targeting signals or domains. The targeting signal can include a sequence of monomers that facilitates in vivo localization of the molecule. The monomers can be amino acids, nucleotide or nucleoside bases, or sugar groups such as glucose, galactose, and the like which form carbohydrate targeting signals. Targeting signals or sequences can be specific for a host, tissue, organ, cell, organelle, non-nuclear organelle, or cellular compartment. For example, in some embodiments the polynucleotide-binding polypeptide includes both a cell-specific targeting domain and an organelle specific targeting domain to enhance delivery of the polypeptide to a subcellular organelle of a specific cells type.

In some embodiments, the polynucleotide-binding polypeptide is modified to target a subcellular organelle. Targeting of the disclosed polypeptides to organelles can be accomplished by modifying the disclosed compositions to contain specific organelle targeting signals. These sequences can target organelles, either specifically or non-specifically. In some embodiments the interaction of the targeting signal with the organelle does not occur through a traditional receptor:ligand interaction.

The eukaryotic cell comprises a number of discrete membrane bound compartments, or organelles. The structure and function of each organelle is largely determined by its unique complement of constituent polypeptides. However, the vast majority of these polypeptides begin their synthesis in the cytoplasm. Thus organelle biogenesis and upkeep require that newly synthesized proteins can be accurately targeted to their appropriate compartment. This is often accomplished by amino-terminal signaling sequences, as well as post-translational modifications and secondary structure.

Organelles can have single or multiple membranes and exist in both plant and animal cells. Depending on the function of the organelle, the organelle can consist of specific components such as proteins and cofactors. The polypeptides delivered to the organelle can enhance or contribute to the functioning of the organelle. Some organelles, such as mitochondria and chloroplasts, contain their own genome. Nucleic acids are replicated, transcribed, and translated within these organelles. Proteins are imported and metabolites are exported. Thus, there is an exchange of material across the membranes of organelles. Exemplary organelles include the nucleus, mitochondrion, chloroplast, lysosome, peroxisome, Golgi, endoplasmic reticulum, and nucleolus. Synthetic organelles can be formed from lipids and can contain specific proteins within the lipid membranes. Additionally, the content of synthetic organelles can be manipulated to contain components for the translation of nucleic acids.
a. Targeting the Mitochondria

In certain embodiments polynucleotide-binding polypeptides are disclosed that specifically target mitochondria. Mitochondria contain the molecular machinery for the conversion of energy from the breakdown of glucose into adenosine triphosphate (ATP). The energy stored in the high energy phosphate bonds of ATP is then available to power cellular functions. Mitochondria are mostly protein, but some lipid, DNA and RNA are present. These generally spherical organelles have an outer membrane surrounding an inner membrane that folds (cristae) into a scaffolding for oxidative phosphorylation and electron transport enzymes. Most mitochondria have flat shelf-like cristae, but those in steroid secreting cells may have tubular cristae. The mitochondrial matrix contains the enzymes of the citric acid cycle, fatty acid oxidation and mitochondrial nucleic acids.

Mitochondrial DNA is double stranded and circular. Mitochondrial RNA comes in the three standard varieties; ribosomal, messenger and transfer, but each is specific to the mitochondria. Some protein synthesis occurs in the mitochondria on mitochondrial ribosomes that are different than cytoplasmic ribosomes. Other mitochondrial proteins are made on cytoplasmic ribosomes with a signal peptide that directs them to the mitochondria. The metabolic activity of the cell is related to the number of cristae and the number of mitochondria within a cell. Cells with high metabolic activity, such as heart muscle, have many well developed mitochondria. New mitochondria are formed from preexisting mitochondria when they grow and divide.

The inner membranes of mitochondria contain a family of proteins of related sequence and structure that transport various metabolites across the membrane. Their amino acid sequences have a tripartite structure, made up of three related sequences about 100 amino acids in length. The repeats of one carrier are related to those present in the others and several characteristic sequence features are conserved throughout the family.

Mitochondrial targeting agents generally consist of a leader sequence of highly positively charged amino acids. This allows the protein to be targeted to the highly negatively charged mitochondria. Unlike receptor:ligand approaches that rely upon stochastic Brownian motion for the ligand to approach the receptor, the mitochondrial localization signal of some embodiments is drawn to mitochondria because of charge. Therefore, in some embodiments, the mitochondrial targeting agent is a protein transduction domain including but not limited to the protein transduction domains discussed in detail above.

Mitochondrial targeting agents also include short peptide sequences (Yousif, et al., Chembiochem., 10(13):2131 (2009), for example mitochondrial transporters-synthetic cell-permeable peptides, also known as mitochondria-penetrating peptides (MPPs), that are able to enter mitochondria. MPPs are typically cationic, but also lipophilic; this combination of characteristics facilitates permeation of the hydrophobic mitochondrial membrane. For example, MPPs can include alternating cationic and hydrophobic residues (Horton, et al., Chem Biol., 15(4):375-82 (2008)). Some MPPs include delocalized lipophilic cations (DLCs) in the peptide sequence instead of, or in addition to natural cationic amino acids (Kelley, et al., Pharm. Res., 2011 Aug 11 [Epub ahead of print]). Other variants can be based on an oligomeric carbohydrate scaffold, for example attaching guanidinium moieties due to their delocalized cationic form (Yousif, et al., Chembiochem., 10(13):2131 (2009).

Mitochondrial targeting agents also include mitochondrial localization signals or mitochondrial targeting signals. Many mitochondrial proteins are synthesized as cytosolic precursor proteins containing a leader sequence, also known as a presequence, or peptide signal sequence. Typically, cytosolic chaperones deliver the precursor protein to mitochondrial receptors and the General Import Pore (GIP) (Receptors and GIP are collectively known as Translocase of Outer Membrane or TOM) at the outer membrane. Typically, the precursor protein is translocated through TOM, and the intermembrane space by small TIMs to the TIM23 or 22 (Translocase of Inner Membrane) at the inner membrane. Within the mitochondrial matrix the targeting sequence is cleaved off by mtHsp70.

As discussed above, in order to enter the mitochondria, a protein generally must interact with the mitochondrial import machinery, consisting of the Tim and Tom complexes (Translocase of the Inner/Outer Mitochondrial Membrane). With regard to the mitochondrial targeting signal, the positive charge draws the linked protein to the complexes and continues to draw the protein into the mitochondria. The Tim and Tom complexes allow the proteins to cross the membranes. Accordingly, one embodiment of the present disclosure delivers compositions of the present disclosure to the inner mitochondrial space utilizing a positively charged targeting signal and the mitochondrial import machinery. In another embodiment, PTD-linked compounds containing a mitochondrial localization signal do not seem to utilize the TOM/TIM complex for entry into the mitochondrial matrix, see Del Gaizo et al. Mol Genet Metab. 80(1-2):170-80 (2003). The N-terminal region of the proteins can be used to target molecules to the mitochondrion. The sequences are known in the art, see for example, U.S. Pat. No. 8,039,587, which is specifically incorporated by reference herein in its entirety. The identification of the specific sequences necessary for translocation of a linked compound into a mitochondrion can be determined using predictive software known to those skilled in the art, including the tools located at http://ihg.gsf.de/ihg/mitoprot.html. Using the software the predicted sequence from Etfa that can be used to target the disclosed composition is MFRAAAPGQL RRAASLLRF (SEQ ID NO:16).

The predicted mitochondrial targeting signal from Dld is MQSWSRVYCS LAKRGHFNRI SHGLQGLSAV PLRTY (SEQ ID NO:17).

In certain embodiments, the mitochondrial targeting agent is the mitochondrial localization signal of a mangano-superoxide dismutase (also referred to herein as “SOD2” and “Mn-SOD” and “superoxide dismutase (Mn)) precursor protein. Several mitochondrial localization signals for SOD2 are known in the art. In some embodiments the mitochondrial targeting signal includes the amino acid sequence

### MLSRAVCGTS RQLPPVLGYL GSRQ (SEQ ID NO:18)

or SEQ ID NO: 18 without the N-terminal methionine

### LSRAVCGTSR QLPPVLGYLG SRQ (SEQ ID NO:19).

In another embodiment the mitochondrial targeting signal includes the amino acid sequence MLSRAVCGTS RQLAPVLGYL GSRQ (SEQ ID NO:20); or SEQ ID NO:20 without the N-terminal methionine LSRAVCGTSR QLAPVLGYLG SRQ (SEQ ID NO:21).

In some embodiments, the composition is preferentially delivered to the mitochondrial using a mitochondrial delivery vehicle, such as a lipid raft, mitochondrially targeted nanoparticle, or mitochondriotropic liposome. In such cases, one or more polynucleotide-binding polypeptides can be associated with, encapsulated within, dispersed in or on, or covalently attached to the mitochondrial delivery vehicle.

In certain embodiments, polynucleotide-binding polypeptides are encapsulated, coupled to, or otherwise associated with mitochondriotropic liposomes. Mitochondriotrophic liposomes are cationic liposomes that can be used to deliver an encapsulated agent to the mitochondria of a cell. Mitochondriotropic liposomes are known in the art. See, for example, U.S. Patent Application Publication No. US 2008/0095834 to Weissig, et al, which is specifically incorporated herein by reference in its entirety. Mitochondriotropic liposomes are liposomes which contain a hydrophobized amphiphilic delocalized cation, such as a triphenylphosphonium or a quinolinium moiety, incorporated into or conjugate to the lipid membrane of the liposome. As a result, the liposomes can be used to deliver compounds incorporated within them to the mitochondria.

In other embodiments, polynucleotide-binding polypeptides are encapsulated within, dispersed in, associated with, or conjugated to a nanoparticle functionalized with one or more mitochondrial targeting agents. For example, the nanoparticle may contain one or be functionalized with one or more lipophilic cations or polypeptide targeting agents.

The nanoparticles may be formed from one or more polymers, copolymers, or polymer blends. In some embodiments, the one or more polymers, copolymers, or polymer blends are biodegradable. Examples of suitable polymers include, but are not limited to, polyhydroxyacids such as poly(lactic acid), poly(glycolic acid), and poly(lactic acid-co-glycolic acids); polycaprolactones; poly(orthoesters); polyanhydrides; poly(phosphazenes); poly(hydroxyalkanoates); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly(alkylene alkylates); hydrophobic polyethers; polyurethanes; polyetheresters; polyacetals ; polycyanoacrylates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), poly(alkylene glycols) such as polyethylene glycol (PEG), poly(propylene glycol) (PPG), and copolymers of ethylene glycol and propylene glycol, poly(oxyethylated polyol), poly(olefinic alcohol), polyvinylpyrrolidone), poly(hydroxy alkylmethacrylamide), poly(hydroxyalkylmethacrylate), poly(saccharides), poly(vinyl alcohol), as well as blends and copolymers thereof. Techniques for preparing suitable polymeric nanoparticles are known in the art, and include solvent evaporation, hot melt particle formation, solvent removal, spray drying, phase inversion, coacervation, and low temperature casting. In some cases, the mitochondrial targeting agents are polypeptides that are covalently linked to the surface of the nanoparticle after particle formulation. In other cases, the mitochondrial targeting agents are lipophilic cations that are covalently bound to the particle surface. In some cases, a cationic polymer is incorporated into the particle to target the particle to the mitochondrion.

Polynucleotide-binding polypeptides can also be targeted to the mitochondria using lipid rafts or other synthetic vesicle compositions. See, for example, U.S. Patent Application Publication No. US 2007/0275924 to Khan, et al. which is specifically incorporated by reference herein in its entirety. The lipid raft compositions can include cholesterol, and one or more lipids selected from the group consisting of sphingomylein, gangliosides, phosphatidylethanolamine, phosphatidylserine, phosphatidylcholine, phosphatidylinositol, and a mitochondrial targeting agent. In certain embodiments, a polypeptide targeting agent is inserted into the lipid raft to target the raft to the mitochondria. The lipid rafts can be prepared and loaded with one or more polynucleotide-binding polypeptides using methods known in the art. See, for example, U.S. Pat. No. 6,156,337 to Barenholz, et al.

A preferred polynucleotide-binding polypeptide that targets mitochondria has at least 80, 85, 90, 95, 99 or 100 percent sequence identity to

(SEQ ID NO: 22)
MARRRRRRRR RRRMAFLRSM WGVLSALGRS GAELCTGCGS
RLRSPFSFVY LPRWFSSVLA SCPKKPVSSY LRFSKEQLPI
FKAQNPDAKT TELIRRIAQR WRELPDSKKK IYQDAYRAEW
QVYKEEISRF KEQLTPSQIM SLEKEIMDKH LKRKAMTKKK
ELTLLGKPKR PRSAYNVYVA ERFQEAKGDS PQEKLKTVKE
NWKNLSDSEK ELYIQHAKED ETRYHNEMKS WEEQMIEVGR
KDLLRRTIKK QRKYGAEEC,

or SEQ ID NO:22 without the N-terminal methionine

(SEQ ID NO: 23)
ARRRRRRRRR RRMAFLRSMW GVLSALGRSG AELCTGCGSR
LRSPFSFVYL PRWFSSVLAS CPKKPVSSYL RFSKEQLPIF
KAQNPDAKTT ELIRRIAQRW RELPDSKKKI YQDAYRAEWQ
VYKEEISRFK EQLTPSQIMS LEKEIMDKHL KRKAMTKKKE
LTLLGKPKRP RSAYNVYVAE RFQEAKGDSP QEKLKTVKEN
WKNLSDSEKE LYIQHAKEDE TRYHNEMKSW EEQMIEVGRK
DLLRRTIKKQ RKYGAEEC.

Another embodiment provides a nucleic acid encoding the polypeptide according to SEQ ID NO:22 is

(SEQ ID NO: 24)
ATGGCGCGTC GTCGTCGTCG TCGTCGTCGT CGTCGTCGTA
TGGCGTTTCT CCGAAGCATG TGGGGCGTGC TGAGTGCCCT
GGGAAGGTCT GGAGCAGAGC TGTGCACCGG CTGTGGAAGT
CGACTGCGCT CCCCCTTCAG TTTTGTGTAT TTACCGAGGT
GGTTTTCATC TGTCTTGGCA AGTTGTCCAA AGAAACCTGT
AAGTTCTTAC CTTCGATTTT CTAAAGAACA ACTACCCATA
TTTAAAGCTC AGAACCCAGA TGCAAAAACT ACAGAACTAA
TTAGAAGAAT TGCCCAGCGT TGGAGGGAAC TTCCTGATTC
AAAGAAAAAA ATATATCAAG ATGCTTATAG GGCGGAGTGG
CAGGTATATA AAGAAGAGAT AAGCAGATTT AAAGAACAGC
TAACTCCAAG TCAGATTATG TCTTTGGAAA AAGAAATCAT
GGACAAACAT TTAAAAAGGA AAGCTATGAC AAAAAAAAAA
GAGTTAACAC TGCTTGGAAA ACCAAAAAGA CCTCGTTCAG
CTTATAACGT TTATGTAGCT GAAAGATTCC AAGAAGCTAA
GGGTGATTCA CCGCAGGAAA AGCTGAAGAC TGTAAAGGAA
AACTGGAAAA ATCTGTCTGA CTCTGAAAAG GAATTATATA
TTCAGCATGC TAAAGAGGAC GAAACTCGTT ATCATAATGA
AATGAAGTCT TGGGAAGAAC AAATGATTGA AGTTGGACGA
AAGGATCTTC TACGTCGCAC AATAAAGAAA CAACGAAAAT
ATGGTGCTGA GGAGTGTTAA.

The sequence encoding the protein transduction domain is underlined, and the sequence encoding the mitochondrial localization signal is double underline. Still another embodiment provides a nucleic acid having at least 80, 85, 90, 95, 99 or more percent sequence identity to SEQ ID NO:24

Another preferred polynucleotide-binding polypeptides that targets mitochondria has at least 80, 85, 90, 95, 97, 99, or 100 percent sequence identity to

(SEQ ID NO: 25)
MRRRRRRRRR RRGEGDIMGE WGNEIFGAIA GFLGGEMLSR
AVCGTSRQLP PVLGYLGSRQ SSVLASCPKK PVSSYLRFSK
EQLPIFKAQN PDAKTTELIR RIAQRWRELP DSKKKIYQDA
YRAEWQVYKE EISRFKEQLT PSQIMSLEKE IMDKHLKRKA
MTKKKELTLL GKPKRPRSAY NVYVAERFQE AKGDSPQEKL
KTVKENWKNL SDSEKELYIQ HAKEDETRYH NEMKSWEEQM
IEVGRKDLLR RTIKKQRKYG AEEC,

or SEQ ID NO:25 without the N-terminal methionine

(SEQ ID NO: 26)
RRRRRRRRRR RGEGDIMGEW GNEIFGAIAG FLGGEMLSRA
VCGTSRQLPP VLGYLGSRQS SVLASCPKKP VSSYLRFSKE
QLPIFKAQNP DAKTTELIRR IAQRWRELPD SKKKIYQDAY
RAEWQVYKEE ISRFKEQLTP SQIMSLEKEI MDKHLKRKAM
TKKKELTLLG KPKRPRSAYN VYVAERFQEA KGDSPQEKLK
TVKENWKNLS DSEKELYIQH AKEDETRYHN EMKSWEEQMI
EVGRKDLLRR TIKKQRKYGA EEC

In another embodiment, the recombinant polypeptide is encoded by a nucleic acid having at least 80, 85, 90, 95, 97, 99, or 100% sequence identity to

(SEQ ID NO: 27)
ATGCGGCGAC GCAGACGTCG TCGTCGGCGG CGTCGCGGCG
AGGGTGATAT TATGGGTGAA TGGGGGAACG AAATTTTCGG
AGCGATCGCT GGTTTTCTCG GTGGAGAAAT GTTATCACGC
GCGGTATGTG GCACCAGCAG GCAGCTGCCT CCAGTCCTTG
GCTATCTGGG TTCCCGCCAG TCATCGGTGT TAGCATCATG
TCCGAAAAAA CCTGTCTCGT CGTACCTGCG CTTCTCCAAA
GAGCAGCTGC CGATTTTTAA AGCGCAAAAT CCGGATGCTA
AAACGACTGA ACTGATTCGC CGCATTGCAC AACGCTGGCG
CGAACTCCCG GACAGTAAAA AAAAAATTTA TCAGGACGCC
TATCGGGCTG AATGGCAGGT CTATAAAGAG GAGATCTCAC
GCTTCAAAGA ACAATTAACC CCGAGTCAAA TAATGTCTCT
GGAAAAAGAA ATCATGGATA AACACTTAAA ACGAAAGGCG
ATGACGAAGA AAAAAGAACT GACCCTGCTA GGTAAACCTA
AGCGTCCGCG CTCTGCGTAT AATGTGTACG TGGCAGAACG
TTTTCAGGAG GCCAAAGGGG ATTCTCCGCA AGAAAAACTG
AAGACCGTCA AAGAAAATTG GAAAAACCTG TCTGATAGCG
AAAAAGAACT GTACATTCAG CACGCTAAAG AAGATGAGAC
GCGGTATCAC AACGAAATGA AATCTTGGGA AGAGCAGATG
ATCGAGGTCG GTCGGAAGGA TCTTCTCCGT CGAACCATCA
AAAAACAGCG TAAATATGGA GCAGAAGAGT GCTGA.

Preferably the mitochondrial targeting signal, domain, or agent does not permanently damage the mitochondrion, for example the mitochondrial membrane, or otherwise impair mitochondrial function.
b. Nuclear Localization Signals

The polynucleotide-binding polypeptides disclosed herein can include one or more nuclear localization signals. Nuclear localization signals (NLS) or domains are known in the art and include for example, SV 40 T antigen or a fragment thereof, such as PKKKRKV (SEQ ID NO:40). The NLS can be simple cationic sequences of about 4 to about 8 amino acids, or can be bipartite having two interdependent positively charged clusters separated by a mutation resistant linker region of about 10-12 amino acids. Additional representative NLS include but are not limited to

(SEQ ID NO: 28)
GKKRSKV;
(SEQ ID NO: 29)
KSRKRKL;
(SEQ ID NO: 30)
KRPAATKKAG QAKKKKLDK;
(SEQ ID NO: 31)
RKKRKTEEES PLKDKAKKSK;
(SEQ ID NO: 32)
KDCVMNKHHR NRCQYCRLQR;
(SEQ ID NO: 33)
PAAKRVKLD;
and
(SEQ ID NO: 34)
KKYENVVIKR SPRKRGRPRK.

3. Additional Sequences

The fusion protein can optionally include additional sequences or moieties, including, but not limited to linkers and purification tags.

In a preferred embodiment the purification tag is a polypeptide. Polypeptide purification tags are known in the art and include, but are not limited to His tags which typically include six or more, typically consecutive, histidine residues; FLAG tags, which typically include the sequence DYKDDDDK (SEQ ID NO:35); haemagglutinin (HA) for example, YPYDVP (SEQ ID NO:36); MYC tag for example ILKKATAYIL (SEQ ID NO:37) or EQKLISEEDL (SEQ ID NO:38). Methods of using purification tags to facilitate protein purification are known in the art and include, for example, a chromatography step wherein the tag reversibly binds to a chromatography resin.

Purifications tags can be N-terminal or C-terminal to the fusion protein. The purification tags N-terminal to the fusion protein are typically separated from the polypeptide of interest at the time of the cleavage in vivo. Therefore, purification tags N-terminal to the fusion protein can be used to remove the fusion protein from a cellular lysate following expression and extraction of the expression or solubility enhancing amino acid sequence, but cannot be used to remove the polypeptide of interest. Purification tags C-terminal to the fusion protein can be used to remove the polypeptide of interest from a cellular lysate following expression of the fusion protein, but cannot be used to remove the expression or solubility enhancing amino acid sequence. Purification tags that are C-terminal to the expression or solubility enhancing amino acid sequence can be N-terminal to, C-terminal to, or incorporated within the sequence of the polypeptide of interest.

In some embodiments, to fusion protein includes one or more linkers or spacers. In some embodiments linker or spacer is one or more polypeptides. In some embodiments, the linker includes a glycine-glutamic acid di-amino acid sequence. The linkers can be used to link or connect two domains, regions, or sequences of the fusion protein.
4. Protein Expression

Molecular biology techniques have developed so that therapeutic proteins can be genetically engineered to be expressed by microorganisms. The gram negative bacterium, Escherichia coli, is a versatile and valuable organism for the expression of therapeutic proteins. Although many proteins with therapeutic or commercial uses can be produced by recombinant organisms, the yield and quality of the expressed protein are variable due to many factors. For example, heterologous protein expression by genetically engineered organisms can be affected by the size and source of the protein to be expressed, the presence of an affinity tag linked to the protein to be expressed, codon biasing, the strain of the microorganism, the culture conditions of microorganism, and the in vivo degradation of the expressed protein. Some of these problems can be mitigated by fusing the protein of interest to an expression or solubility enhancing amino acid sequence. Exemplary expression or solubility enhancing amino acid sequences include maltose-binding protein (MBP), glutathione S-transferase (GST), thioredoxin (TRX), NUS A, ubiquitin (Ub), and a small ubiquitin-related modifier (SUMO).

In some embodiments, the compositions disclosed herein include expression or solubility enhancing amino acid sequence. In some embodiments, the expression or solubility enhancing amino acid sequence is cleaved prior administration of the composition to a subject in need thereof. The expression or solubility enhancing amino acid sequence can be cleaved in the recombinant expression system, or after the expressed protein in purified. In some embodiments, the expression or solubility enhancing is a ULP1 or SUMO sequence. Recombinant protein expression systems that incorporate the SUMO protein (“SUMO fusion systems”) have been shown to increase efficiency and reduce defective expression of recombinant proteins in E. coli., see for example Malakhov, et al., J. Struct. Funct. Genomics, 5: 75-86 (2004), U.S. Pat. No. 7,060,461, and U.S. Pat. No. 6,872,551. SUMO fusion systems enhance expression and solubility of certain proteins, including severe acute respiratory syndrome coronavirus (SARS-CoV) 3CL protease, nucleocapsid, and membrane proteins (Zuo et al., J. Struct. Funct. Genomics, 6:103-111 (2005)).

B. Combination Therapies

In some embodiments the compositions including an effective amount of the fusion proteins disclosed herein are administered in combination with one or more second therapeutic agents. For example, the composition itself can include a combination of a polynucleotide-binding polypeptide and one or more second therapeutic agents. In another embodiment, a first composition including a polynucleotide-binding polypeptide is co-administered with one or more additional compositions including one or more second therapeutic agents.

In certain embodiments, the second therapeutic agent is a conventional therapeutic for treating one or more symptoms side effects associated with therapeutic administration of an antineoplastic agent in subject or exposure to high levels of radiation, including, but not limited to, vitamin supplements, appetite-stimulating medications, medications that help food move through the intestine, nutritional supplements, anti-anxiety medication, anti-depression medication, anti-coagulants, clotting factors, antiemetic medications, antidiarrheal medications, anti-inflammatories, steroids such as corticosteroids or drugs that mimic progesterone, omega-3 fatty acids supplements, and eicosapentaenoic acid supplements.

### EXAMPLES

### Example 1

### TFAM Reduces the Toxicity of Doxorubicin

### Materials and Methods

“PTD−TFAM”, “TFAM”, and “rhTFAM” as used in this experiment is a fusion protein with a protein transduction domain, a mitochondrial localization signal, and a TFAM polypeptide.

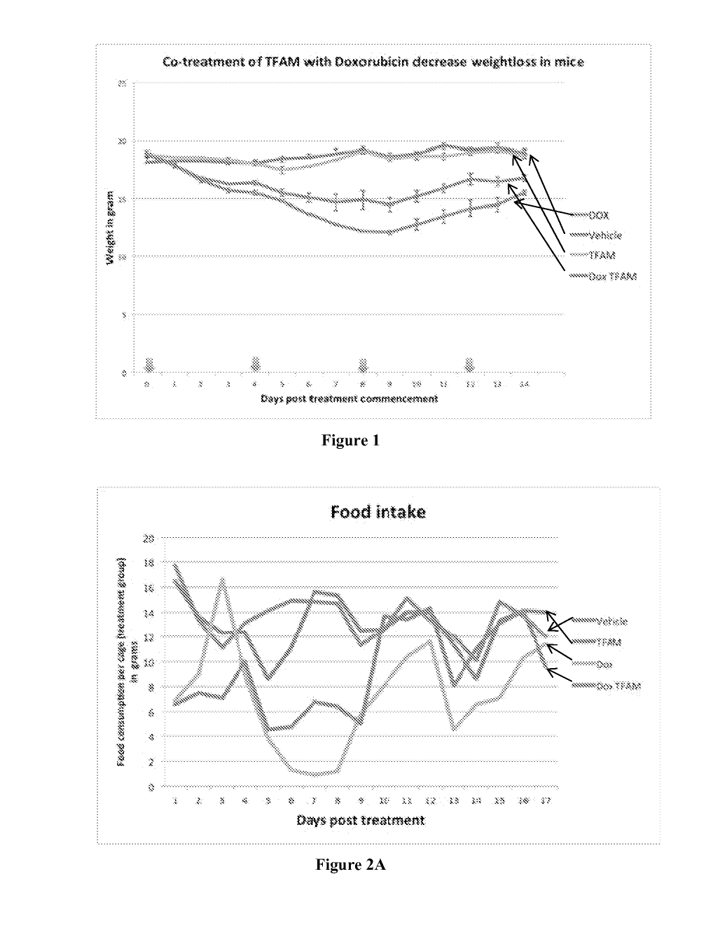

Two of four groups of five naive C57/BL6 mice were treated with doxorubicin (dox) at dose of 25 mg/kg, whereas the other two groups were left untreated. To assess PTD−TFAM's ability to ameliorate the toxic effects of Doxorubicin one group each of the dox-treated and untreated animals received PTD−TFAM at a dose of 0.3mg/kg on the same day as they were treated with Doxorubicin and every four days for two weeks thereafter (indicated with arrows in FIG. 1). Concurrently mice in the last two groups (one treated and one untreated with Doxorubicin) received vehicle alone (100 μl of 50% sorbitol, 2×PBS). To assess toxicity of the Doxorubicin treatment, mice were weighed once daily.

Each treatment group was housed together in cage separate from the other treatment group(s). The animals in each case share a food hopper. Food intake was evaluated on a group-by-group basis by weighing each group's food once daily. The difference in food weight between two days provides a rough estimate of the group's food intake during the period between two measurements (i.e, a day).

### RESULTS

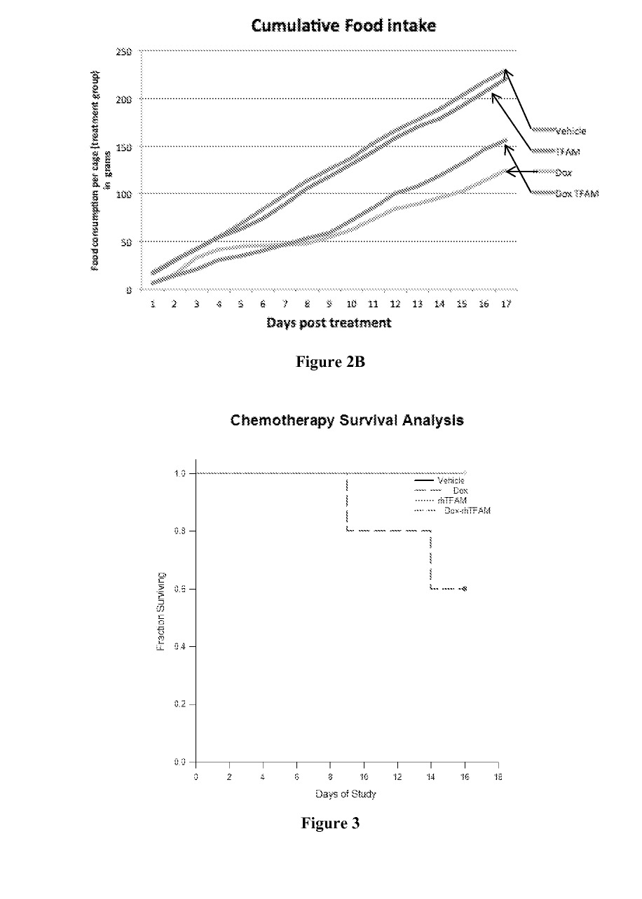

Body weight, food intake and survival are three indications of the toxicity used to evaluate the health and condition of mice in each treatment group. In mice treated with dox only, body weight decreased significantly starting immediately after dosing, and continuing for up to ten days (FIG. 1). Within five days the mice in the dox only treatment group decreased their food intake sharply and had low food intake during days 6, 7 and 8 (FIG. 2). During days 6-8 the mice in the dox only treatment group consumed less than two grams of food combined, compared to vehicle treated animals which consumed more than ten-fold this amount (FIG. 2). Starting on day nine, the food intake of the mice in the dox only treatment group increased sharply, and their bodyweights began to recover accordingly (FIGS. 1 and 2). One of the mice in dox only treatment group began to gain weight, but died on day 14 (FIG. 3). It is believed that the mouse died from heart failure due to oxidative damage to myocardium.

Co-administration of PTD−TFAM with dox reduced the toxic effect of dox treatment alone. The mice of the PTD−TFAM+dox treatment group lost less body weight, and their food intake did not drop as low as the dox only treatment group (FIGS. 1 and 2). No deaths were observed in PTD−TFAM+dox treatment group (FIG. 3).

Mice treated with PTD−TFAM+dox showed higher body weight, greater food intake, and higher survival compared to the dox only treatment group, indicating that mice treated with PTD−TFAM+dox are better able to tolerate a high dose of doxorubicin than mice treated with dox only.

### Example 2

### PTD−TFAM Reduces the Toxicity of Gemcitabine in an Orthotopic Model of Pancreatic Cancer

### Materials and Methods

“PTD−TFAM”, “TFAM”, and “rhTFAM” as used in this experiment is a fusion protein with a protein transduction domain, a mitochondrial localization signal, and a TFAM polypeptide.

MiaPaCa-2 tumor cells was maintained in vitro as monolayer culture in DMEM medium supplemented with 10% fetal bovine serum, 2.5% horse serum, 100 U/ml penicillin and 100 μg/m1 streptomycin, and 2 mM L-glutamine at 37° C. in an atmosphere of 5% CO₂ in air. The tumor cells were routinely subcultured twice weekly by trypsin-EDTA treatment. Cells in an exponential growth phase will be harvested and counted for tumor inoculation. Before orthotopic implantation, 5×10⁶ MiaPaca-2 cells in 50 μl PBS mixed with 50 μl Matrigel were inoculated subcutaneously at the right flank of each BALB/c nude mouse. Upon subcutaneous tumor size reaching about 500 mm³, tumors were collected for orthotopic implantation.

For orthotopic MiaPaca-2 xenografts, mice were anesthetized by intraperitoneal injection of pentobarbital sodium (50 mg/kg) before tumor inoculation. The abdominal skin was sterilized and laparotomy was performed to expose the pancreas. Each mouse was inoculated with a subcutaneous MiaPaca-2 tumor fragment (2-3 mm in diameter) in the subcapsular region of the pancreas for tumor development. The abdominal wall was closed using No. 6 suture and then sterilized with povidone iodine solution. The day of surgery was denoted as Day 0 (DO). The treatments with vehicle, gemcitabine only, PTD−TFAM only, and PTD−TFAM in combination with gemcitabine (TFAM+gem) were started at 10 days post tumor fragment inoculation. The day was denoted as Day 1 of treatment (PG-D1). PTD−TFAM was administered at a dose of 0.15mg/kg every fourth day commencing at day PG-D1. Gemcitabine was administrated at a dose of 40 mg/kg bodyweight every 3 days, starting at day PG-D1. The body weight of each mouse was measured on treatment days 1, 5, 8, 12, 15, 19 and 22. The experiment was terminated on day 22.

### RESULTS

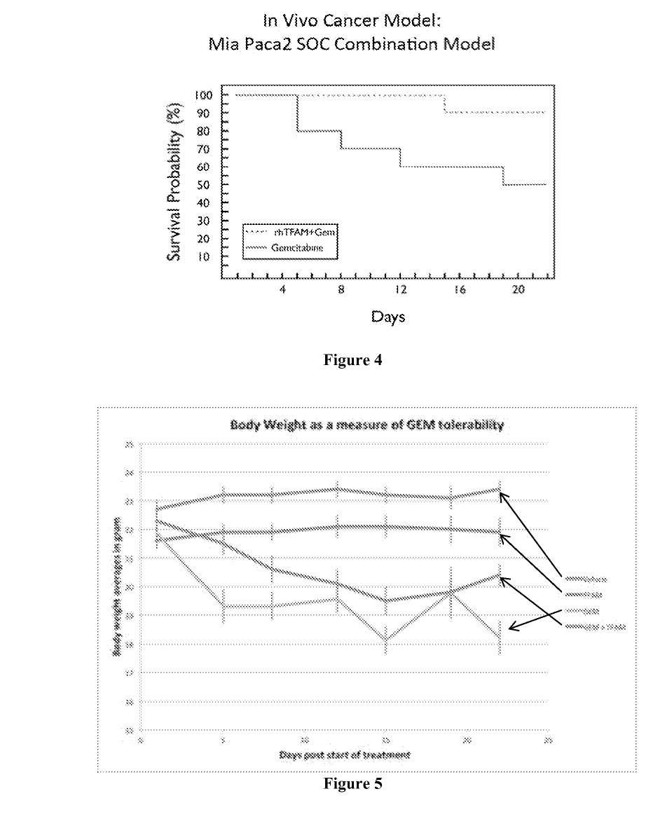

Gemcitabine treatment exhibited high toxicity as observed by diminished survival (FIG. 4), and strongly decreased body weights (FIG. 5). The toxicity was reduced by co-treatment with PTD−TFAM. As shown in FIG. 4, by day 22, 50% of the mice in gemcitabine only treatment group were dead, compared only one out of ten mice in the TFAM+gem treatment group. Tumor growth inhibition differences between the different treatment groups were not statistically significant indicating that PTD−TFAM did not reduce the anti-tumor activity of gemcitabine.

### Example 3

### rhTFAM Increases the Survival and Weight Gain of C3H/HeN Mice Following Total Body Irradiation

### Materials and Methods

“PTD−TFAM”, “TFAM”, and “rhTFAM” as used in this experiment is a fusion protein with a protein transduction domain, a mitochondrial localization signal, and a TFAM polypeptide.

Thirty-two male C3H/He N Mice (Charles River Laboratories) were used in the study. The animals were fed with Labdiet 5053 Rodent chow and water ad libitum aged 6 to 7 weeks. All animals in all groups exhibited similar body weights of approximately 22-24 g at the commencement of the study.

Animals were individually numbered using an ear punch and housed in small groups of approximately 16 animals per cage. Animals were acclimatized for at least 3 days prior to study commencement, during which the animals were observed daily and confirmed to be in good health prior to treatment.

The mice were given an acute total body radiation dose of 650 cGy on day 0. Radiation was generated with a 160 kilovolt potential (18-ma) source at a focal distance of 50 cm, hardened with a 0.35 mm Al filtration system. Irradiation was targeted the total body at a rate of <100 cGy/minute. Survival and weights was evaluated daily as an indication for radiation induced toxicity until the conclusion of the experiment on day 30.

Each treatment group was housed together in cage separate from the other treatment group(s). The animals in each case share a food hopper. Food intake was evaluated on a group-by-group basis by weighing each group's food once daily. The difference in food weight between two days provides a rough estimate of the group's food intake during the period between two measurements (i.e, a day).

On day 0, mice were exposed to radiation in a pie cage to a total dose of 650 cGy. No anesthesia was used during irradiation. Animals were dosed with vehicle or 0.35 mg/kg/dose rhTFAM via intravenous injection (iv) once every four days, with the first dose of test article administered 4 hours after irradiation on day 0 and subsequent doses administered on day 4, 8, 12, 16, 20, 24 and 28.

### RESULTS

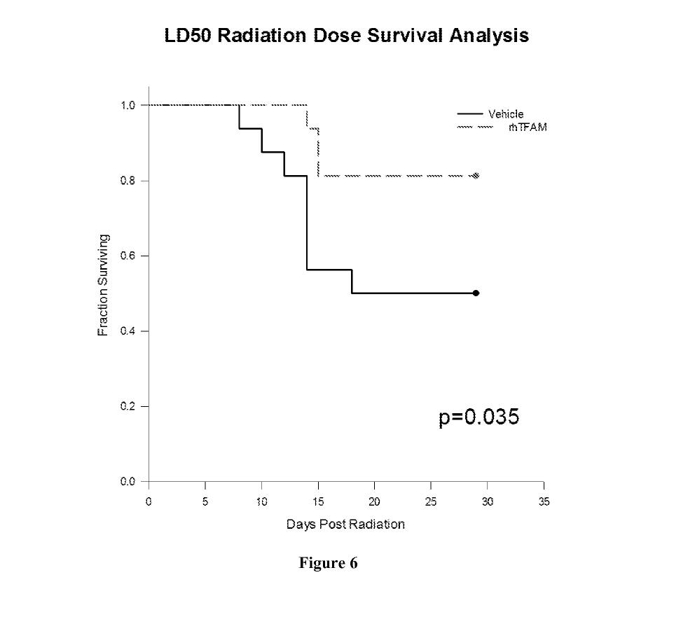

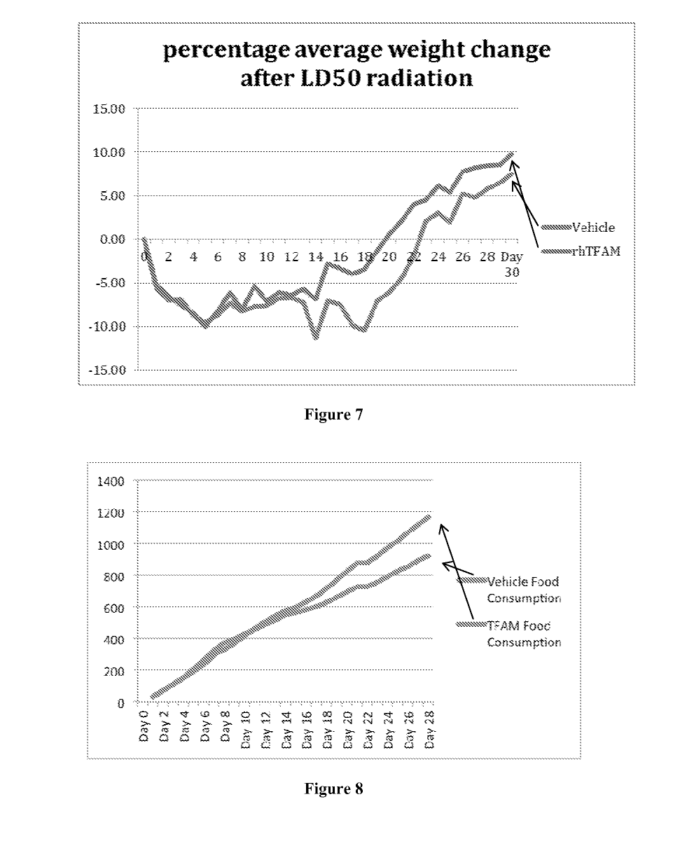

13/16 (81%) of rhTFAM treated animals survived the acute total body radiation, whereas only 8/16 (50%) of the vehicle treated animals survived (FIG. 6). Although the animals from both vehicle and rhTFAM treatment groups suffered about the same acute weight loss after radiation exposure (about 10% below their initial weight on day 5), the animals in the rhTFAM treated group regained their weight more quickly than vehicle treated mice Animals in the rhTFAM treated group were 5% below their initial weight on day 15, and were back to their initial weight on day 19 (FIG. 7). Vehicle treated animals had a delayed recovery and were not back up to 5% below their initial weight until day 21, almost one full week later than the rhTFAM treated group. After day 21, animals in the vehicle treated group gained weight quickly and were back to their initial weight on day 22. Food intake did not differ between the two groups during the first 15 days, the period of highest toxicity as measured by mortality (FIGS. 6 and 8). In the second half of the study, food intake was elevated by 30-50% in the rhTFAM treated group.

### Example 4

### rhTFAM Lowers the 1050 Concentration for Five Different Chemotherapeutic Agents Cytokine Levels in Tumor Xenografts

### Materials and Methods

Approximately 1500 PanO2 murine pancreatic adenocarcinoma cells were plated in a 96 well plate, and placed in a hypoxia chamber, which was purged with nitrogen for 15 minutes. The chamber was sealed and placed in 37° C. incubator without CO₂ for 5 days. Plates were removed and inspected visually under a phase contrast light microscope.

Cells were treated with vehicle control or 10 nM rhTFAM, and various dosages of Gem: Gemcitabine (0.05 μM, 0.025 μM, or 0.0125 μM) TMZ: Temozolamide (50 μM, 25 μM, or 1.25 μM), Dox: Doxorubicin (Adriamycin) (1.25 μM, 0.625 μM, or 0.3125 μM), Cis: Cisplatin (25 μM, 12.5 μM, or 6.25 μM), or 2-DG: 2-deoxy glucose (25 μM, 12.5 μM, or 6.25 μM).

rhTFAM and drugs were added to cells upon plating and left on the cells for the duration of the experiment (i.e., 5 days). The cells were left in a hypoxia chamber and not disturbed to keep oxygen levels low.

Cells were subsequently prepared for live/dead testing according to manufacturer's protocol (Life Technologies #L3224).

### RESULTS

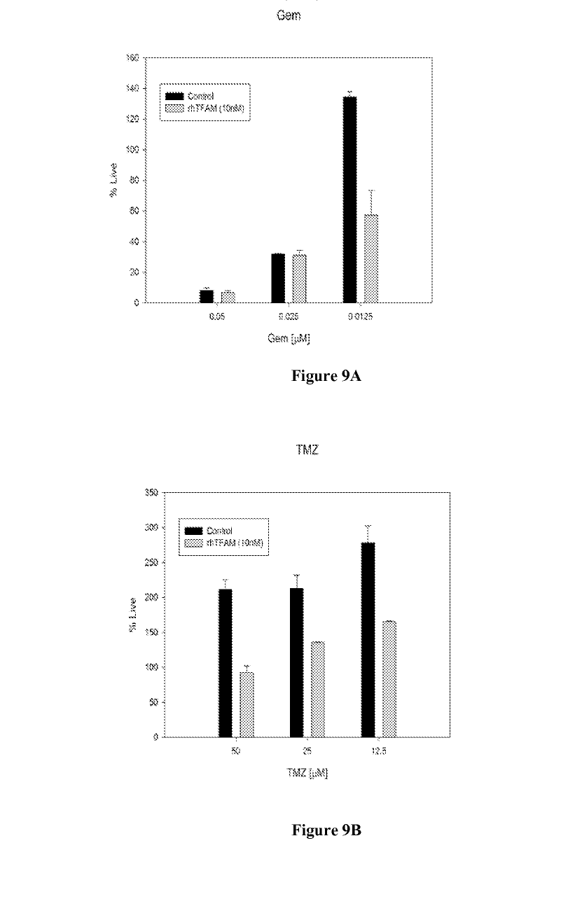

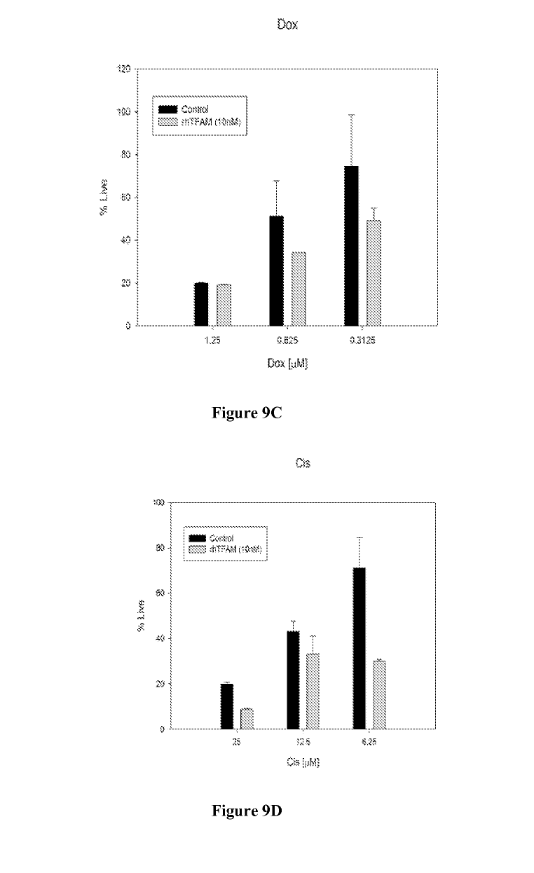

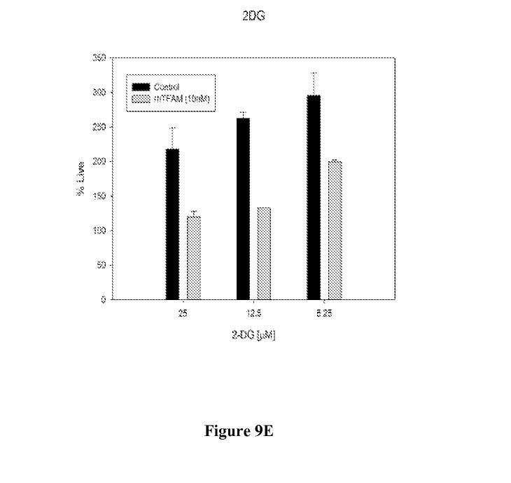

rhTFAM sensitizes cancer cells to chemotherapeutic drugs under hypoxic conditions as shown in FIG. 9A-E, Gem: Gemcitabine, TMZ: Temozolamide, Dox: Doxorubicin (Adriamycin), Cis: Cisplatin, 2-DG: 2-deoxy glucose.

### Example 5

### rhTFAM Increases Oxygen Consumption Rate in Cancer Cells

### Materials and Methods

Using an Extracellular Flux Analyzer, glycolysis and mitochondrial respiration were analyzed in four different breast cancer cell lines (MCF7, HCC202, MDA-MB-231, BT-474) representing four subtypes (HER2− ER+, HER2+ ER−, HER2− ER−, HER2+, ER+). Measurements were taken using the Seahorse XF 24 instrument. Adherent cells were seeded according to its growth rate in 24 well cell culture microplates and cultured overnight. Approximately 30 min prior to assay, culture medium was exchanged for unruffled Seahorse assay medium. Bioenergetic differences among the cell lines were assayed by measuring Oxygen Consumption Rate (OCR) and Extracellular Acidification Rate (ECAR) in response to rhTFAM (1 nM) treatment.

### RESULTS

An experiment was designed to test the effect of rhTFAM on the metabolic signature of cells. The results of the experiment are summarized in Table 2 below.

TABLE 2
rhTFAM Increases Oxygen Consumption Rate in Cancer Cells

| Cell Line | OCR | ECAR | OCR/ECAR |
|---|---|---|---|
| MCF-7 | 11 ± 2 | −41 ± 3 | 188 ± 7.5 |
| MDA-MB-231 | 7 | −13 | 123 |
| BT 474 | 17 ± 2 | −71 ± 4 | 403 ± 62.9 |
| HCC-202 | 13 ± 1 | −37 ± 2 | 179 ± 7.5 |

The results show that treatment of various cancer cell lines with rhTFAM in vitro causes an increase of oxygen consumption rate (OCR) and a concomitant decrease in extracellular acidification rate (ECAR) (% over non-treated control). Results are presented as percentage points increase or decrease compared to control. OCR/ECAR values are shown as OCR/ECAR ratios expressed as percentage (i.e. MCF-7 111/59=1.88). Extracellular acidification rate (ECAR) is an indirect measure of glycolysis. MCF-7, MDA-MB-231, BT 474 and HCC-202 are breast cancer cell lines. These data support the conclusion that rhTFAM induces a metabolic switch from glycolysis to oxidative phosphorylation in cancer cells.

### Example 6

### rhTFAM Lowers Delta-Psi in Cancer Cells, Sensitizing the Cells to Apoptosis

### Materials and Methods

The mitochondrial potentiometric dye, JC-1, was utilized to assay relative changes in mitochondrial membrane potential (Δψm). A 96-well black culture plate was seeded with non-malignant human fibroblasts and HepG2, a human hepatocellular carcinoma cell line at 1×10⁶ cells/well twenty-four hours before the experiment was begun. Cells were maintained in 100 μl culture medium per well in a CO₂ incubator overnight at 37° C. Cells were treated with rhTFAM at 1-2 ug/mL or left untreated in triplicate. 10 μl of the JC-1 Staining Solution was added to each well and mixed gently. The cells were incubated in a CO₂ incubator at 37° C. for 15 minutes. The cell media was aspirated and additional 200 uL of cell media was added. This was repeated two times. 100 μl of cell media was added to each well. The cells were analyzed in fluorescent plate reader. In healthy cells, JC-1 forms J-aggregates which display strong fluorescent intensity with excitation and emission at 560 nm and 595 nm, respectively. In apoptotic or unhealthy cells, JC-1 exists as monomers which show strong fluorescence intensity with excitation and emission at 485 nm and 535 nm, respectively. The ratio of fluorescent intensity of J-aggregates to fluorescent intensity of monomers can be used as an indicator of mitochondrial membrane potential.

### RESULTS

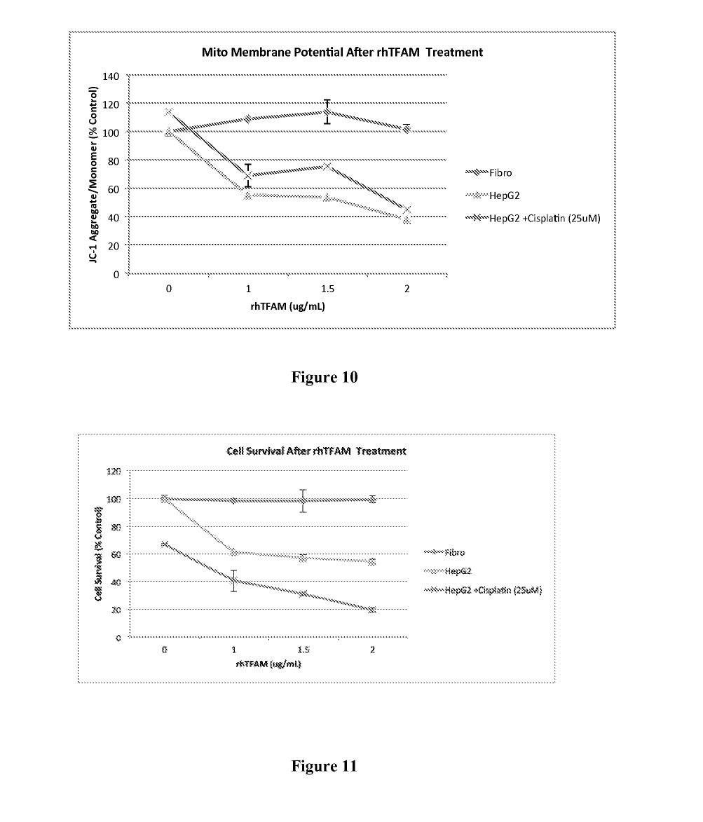

An experiment was designed to test the effect of rhTFAM on mitochondrial membrane potential. The results presented in FIG. 10 indicate a decrease in mitochondrial potential in HepG2 tumor cells but not in non-malignant fibroblast cells (Fibro) after addition of rhTFAM.

### Example 7

### rhTFAM Reduces Survival of Cancer Cells

### Materials and Methods

The Cell-Titre Glo (Promega) cell survival assay kit was utilized. Briefly, a 96-well black culture plate was seeded with human fibroblasts and HepG2 cells at 1×10⁶ cells/well twenty-four hours before the experiment was begun. Cells were maintained in 100 μl culture medium per well in a CO₂ incubator overnight at 37° C. Cells were treated with rhTFAM at 1-2 ug/mL or left untreated in triplicate for 48 hours. Cell-Tire Glo reagents were thawed and mixed and 100 uL of the mix added to each well. Plates were transferred to a plate reader for luminescent reads. The plate was mixed by orbital shaking for 2 minutes followed by 10 minutes at room temperature at the conclusion of which luminescence was measured and expressed in terms of vehicle control.

### RESULTS

An experiment was designed to test the effect of rhTFAM on apoptosis. The results of the experiment are presented in FIG. 11 which shows that rhTFAM treatment increases cell death via caspase activation and PARP cleavage.

### Example 8

### rhTFAM Treatment Induces Tumor Growth Inhibition (TGI) In Vivo

### Materials and Methods

A pancreatic carcinoma cell line (Mia PaCa 2), a gliobastoma cell line (U-87) a breast carcinoma cell line (MCF-7), a hepatocellular carcinoma (HepG2), a prostate carcinoma (DU-145), and a melanoma cell line (B16F10) were utilized in xenograft experiments. Eight weeks old immunosuppressed (nude) mice maintained under sterile conditions were injected subcutaneously in the right flank with 5×10⁶ cells one the respective cell lines suspended in 100 μl of matrigel. MCF-7 injected mice also received seventeen β-estradiol pellets implanted subcutaneously around the left forearm using a trochar. When the tumor size reached approximately 100 mm³ volume, the mice were divided into five groups of eight mice each and dosing was initiated. The mice were treated every four days with vehicle (50% sorbitol 233 PBS), or 0.33, 0.5, 0.66 or 1.0 mg/kg of rhTFAM. Tumor sizes were measured twice a week and at the end of the study the median size of tumors of the control arm was compared to the median size of tumors.

### RESULTS

An experiment was designed to test the effect of rhTFAM on tumors in vivo. Table 3 lists the tumor growth inhibition (TGI) from the respective treatment groups were rhTFAM was most efficacious, which tended to be the low dose in most experiments. The size of the tumors is expressed as a percentage of decrease of the size of the tumors in non-treated control groups as shown in Table 3 below.

TABLE 3
rhTFAM Treatment Induces Tumor
Growth Inhibition (TGI) in vivo

| Tumor Model | TGI (Single Agent) |
|---|---|
| HepG2 (Liver) | 58% |
| DU-145 (Prostate) | 57% |
| MCF-7 (Breast) | 40% |
| U87 (Brain) | 34% |
| Mia Paca-2 (Pancreas) | 70% |
| B16F10 (Melanoma) | 25% |

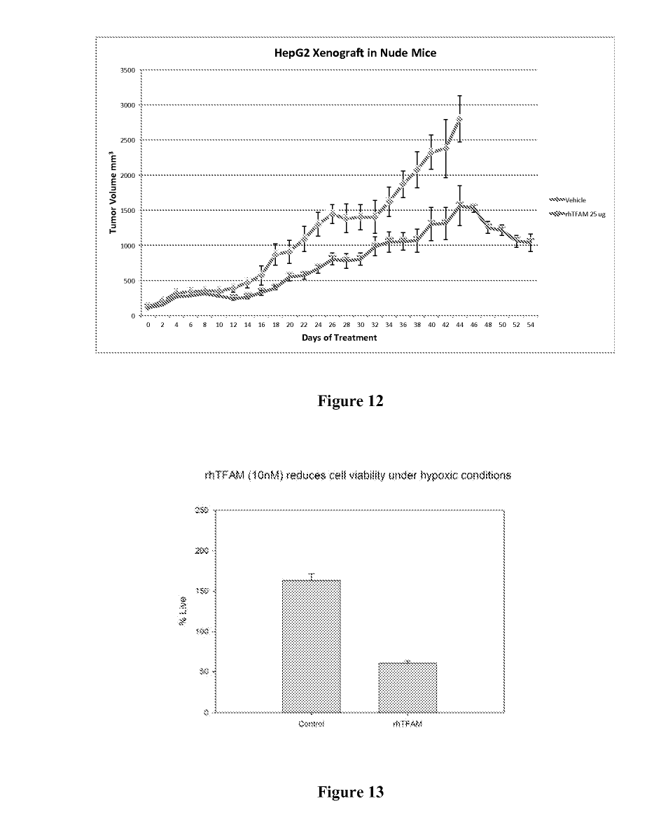

By means of example, the full data set from the HepG2 tumor measurements is shown in the FIG. 12, which shows that rhTFAM Treatment induces Tumor Growth Inhibition (TGI) in HepG2 xenograph model.

### Example 9

### rhTFAM Reduces Survival of Cancer Cells Under Hypoxic Conditions

### Materials and Methods

1,500 PanO2 murine pancreatic adenocarcinoma cells were plated in designated wells on 96 well plate and incubated without oxygen (100% nitrogen gas), at 37° C. for five days. After visual inspection under a light microscope, cells were washed twice in PBS and the dyes were added to the cells and incubated 30-45 minutes according to the manufacturer's protocol (Life Technologies #L3224) and analyzed on a BMG Pherastarm plate reader.

### RESULTS

Many cancer agents do not work well under hypoxia. Therefore, an experiment was designed to test the effect of rhTFAM on cancer cell survival under hypoxic conditions. The results of the experiment are presented in FIG. 13 which shows that rhTFAM decreases cancer cell survival under hypoxic conditions.

The data presented in Examples 5-9 shows that rhTFAM treatment (i) increases oxygen consumption in cancerous cell lines (Example 5); (ii) decreases Δψm in cancerous cells, but not in non-cancerous cells (Example 6); (iii) increases apoptosis in cancerous cell lines (Example 7); (iv) decreases tumor growth in mice (Example 8); and (v) decreases cancer cells survival under hypoxic conditions (Example 9).

1. A method of reducing the toxicity of chemotherapy in a subject comprising administering to the subject a fusion protein comprising a protein transduction domain, a targeting signal and a transcription factor A—mitochondrial (TFAM) polypeptide in an amount effective to inhibit, reduce or alleviate one or more side effects of the chemotherapy or radiation therapy.

2. (canceled)

3. A method of reducing the toxicity of occupational, accidental, or weapon-induced exposure to radiation in a subject comprising administering to the subject a fusion protein comprising a protein transduction domain, a targeting signal and a transcription factor A—mitochondrial (TFAM) polypeptide in an amount effective to inhibit, reduce or alleviate one or more side effects of the exposure to radiation.

4. A method for treating side effects associated with administering to a subject a therapeutic dose of an antineoplastic agent comprising administering to the subject a fusion protein comprising a protein transduction domain, a targeting signal and a transcription factor A—mitochondrial (TFAM) polypeptide in an amount effective to inhibit, reduce or alleviate one or more side effects of the antineoplastic agent compared to a control.

5. The method of claim 4 wherein the antineoplastic agent is one or more chemotherapeutic drugs, radiation, or a combination thereof.

6. The method of claim 5 wherein the chemotherapeutic drug is selected from the group consisting of alkylating agents, antimetabolites, antimitotics, anthracyclines, cytotoxic antibiotics, and topoisomerase inhibitors.

7. The method of claim 6 wherein the antimetabolite is gemcitabine.

8. The method of claim 6 wherein the anthracycline is doxorubicin.

9. The method of claim 1 wherein the side effect is selected from the group consisting of reduced appetite (i.e., food intake), weight loss, myelosuppression, mucusitis, low red blood cells count (anemia), fatigue, constipation, diarrhea, nausea and vomiting, bleeding problems, hair loss (alopecia), infection, memory changes, mouth and throat changes, nerve changes, pain, sexual and fertility changes, skin and nail changes, swelling (fluid retention), urination changes (including changes in color and frequency), flu-like symptoms, low infection-fighting white blood cells count (neutropenia), low platelets count (thrombocytopenia). Other side effects include malnutrition, dehydration, damage to the heart, liver, kidney, inner ear and brain, abdominal pain, impotence, acid indigestion, incoordination, acid reflux, infection, allergic reactions, injection site reactions, alopecia, injury, anaphylasix, insomnia, anemia, iron deficiency anemia, anxiety, itching, lack of appetite, arthralgias, asthenia, joint pain, ataxia, azotemia, kidney problems, balance & mobility changes, bilirubin blood level, leukopenia, bone pain, loss of libido, bladder problems, liver dysfunction, bleeding problems, liver problems, blood clots, loss of libido, blood pressure changes, low blood counts, blood test abnormalities, low blood pressure (hypotension), breathing problems, low platelet count, bronchitis, low red blood cell count, bruising, low white blood cell count, lung problems, cardiotoxicity, cardiovascular events, memory loss, cataracts, menopause, central neurotoxicity, metallic taste, chemo brain, mouth sores, chest pain, mucositis, chills, muscle pain, cognitive problems, myalgias, cold symptoms, myelosuppression, confusion, myocarditis, conjunctivitis (pink eye), constipation, cough, nail changes, cramping, nausea, cystitis, nephrotoxicity, nervousness, neutropenia, deep vein thrombosis (DVT), neutropenic fever, dehydration, nosebleeds, depression, numbness, diarrhea, dizziness, drug reactions, ototoxicity, dry eye syndrome, dry mouth, dry skin, pain, dyspepsia, palmar-plantar erythrodysesthesia (PPE), dyspnea, pancytopenia, pericarditis, peripheral neuropathy, early satiety, pharyngitis, edema, photophobia, electrocardiogram (ECG/EKG) changes, photosensitivity, electrolyte imbalance, pneumonia, esophagitis, pneumonitis, eye problems, post-nasal drip, proteinuria, pulmonary embolus (PE), fatigue, pulmonary fibrosis, feeling faint, pulmonary toxicity, infertility, fever, flatulence, radiation recall, flu-like syndrome, rash, flushing, rapid heart beat, rectal bleeding, restlessness, gas, rhinitis, gastric reflux, ringing ears, gastroesophageal reflux disease (GERD), runny nose, genital pain, granulocytopenia, gynecomastia, sadness, glaucoma, seizures, safety, hyposexuality, shortness of breath, hair loss, sinusitis, hand-foot syndrome, skin reactions, headache, sleep problems, hearing loss, sore mouth, hearing problems, stomach sour, heart failure, stomach upset, heart palpitations, stomatitis, heart problems, swelling, heart rhythm changes, heartburn, hematoma, taste changes, hemorrhagic cystitis, thrombocytopenia, hepatotoxicity, low thyroid hormone levels, high blood pressure (hypertension), tingling, high liver enzymes, tinnitus, hyperamylasemia (high amylase), trouble sleeping, hypercalcemia (high calcium), hyperchloremia (high chloride), hyperglycemia (high blood sugar), urinary tract infection, hyperkalemia (high potassium), hyperlipasemia (high lipase), hypermagnesemia (high magnesium), vaginal bleeding, hypernatremia (high sodium), vaginal dryness, hyperphosphatemia (high phosphous), vaginal infection, hyperpigmentation, vertigo, hypersensitivity skin reactions, vomiting, hypertriglyceridemia (high triglycerides), hyperuricemia (high uric acid), hypoalbuminemia (low albumin), water retention, hypocalcemia (low calcium), watery eyes, hypochloremia (low chloride), weakness, hypoglycemia (low blood sugar), weight changes, hypokalemia (low potassium), weight gain, hypomagnesemia (low magnesium), weight loss, hyponatremia (low sodium), hypophosphatemia (low phosphous) and xerostomia death. In the most preferred embodiments, the composition is administered in an effective amount to increase appetite (i.e., food intake), reduce weight loss, death or combinations thereof.

10. The method of claim 1 wherein the composition is administered prophylactically, therapeutically, or a combination thereof.

11. The method of claim 1 wherein the protein transduction domain comprises between 7-15 arginine residues.

12. The method of claim 1 wherein the targeting signal comprises an organelle targeting signal.

13. The method of claim 12 wherein the organelle targeting signal is a mitochondrial localization signal.

14. The method of claim 13 wherein the mitochondrial localization signal comprises the mitochondrial localization signal of a SOD2.

15. The method of claim 1 wherein the TFAM polypeptide comprises at least one HMG box of TFAM.

16. The method of claim 1 wherein the TFAM polypeptide comprises a fragment of TFAM effective to increase oxidative phosphorylation in a mitochondrion.

17. The method of claim 1 wherein the TFAM polypeptide comprises mature human TFAM.

18. The method of claim 1 wherein the recombinant fusion protein comprises SEQ ID NO:26, or a variant thereof with 90% sequence identify over the full-length of SEQ ID NO:26.
